(19) United States
(12) Patent Application Publication
(10) Pub. No.: US 2023/0028710 A1
(43) Pub. Date: Jan. 26, 2023

(54) IDENTIFICATION OF MICROBIAL CONTAMINATIONS OR INFECTIONS IN LIQUID SAMPLES BY RAMAN SPECTROSCOPY

(71) Applicant: microPhotonX GmbH, Tutzing (DE)

(72) Inventors: Karin Schütze, Tutzing (DE); Raimund Schütze, Tutzing (DE); Hesham Kamaleldin Aly Mahmoud Yosef, Penzberg (DE)

(73) Assignee: microPhotonX GmbH, Tutzing (DE)

(21) Appl. No.: 17/788,347

(22) PCT Filed: Dec. 22, 2020
(86) PCT No.: PCT/EP2020/087662

§ 371 Date: Jun. 23, 2022

(30) Foreign Application Priority Data
Dec. 23, 2019 (GB) 1919190.7

(51) Int. Cl. G01N 21/65; G01N 1/40; C12Q 1/18; B01L 3/00

(52) U.S. Cl. CPC G01N 21/65; G01N 1/4077; C12Q 1/18; B01L 3/502753; G01N 2001/4083; B01L 2300/0681; B01L 2200/16; B01L 2300/0809

(57) ABSTRACT

The present invention relates to vitro method for analysing a liquid sample as to the presence, identity and properties of microbes comprising: a) isolating microbes from the liquid sample; b) analysing said microbes spectroscopically by means of spontaneous Raman spectroscopy; and c) determining antibiotic susceptibility of said microbes spectroscopically by means of spontaneous Raman spectroscopy. The present invention also refers to device for analysing a liquid sample as to the presence, identity and properties of microbes, wherein the device comprises as a first unit (i) a chip comprising a filtering unit and an antibiotics exposure unit capable of determining the susceptibility of microbes to an antibiotic; as a second unit (ii) a Raman spectroscopy system; and as a third unit (iii) an evaluation module which is coupled to the Raman spectroscopy system.

### FIELD OF THE INVENTION

The present invention relates to vitro method for analysing a liquid sample as to the presence, identity and properties of microbes comprising: a) isolating microbes from the liquid sample; b) analysing said microbes spectroscopically by means of spontaneous Raman spectroscopy; and c) determining antibiotic susceptibility of said microbes spectroscopically by means of spontaneous Raman spectroscopy. The present invention also refers to device for analysing a liquid sample as to the presence, identity and properties of microbes, wherein the device comprises as a first unit (i) a chip comprising a filtering unit and an antibiotics exposure unit capable of determining the susceptibility of microbes to an antibiotic; as a second unit (ii) a Raman spectroscopy system; and as a third unit (iii) an evaluation module which is coupled to the Raman spectroscopy system.

### BACKGROUND OF THE INVENTION

Early detection and rapid identification of microbes in liquid specimens are indispensable for successful treatment of patients with microbial infections. In particular in the case of sepsis and other infections which are associated with high morbidity and mortality in spite of the broad range of antibiotics, fast and reliable detection of microorganisms in biological fluids and of their susceptibility to antibiotics or bacteriocidal substances such as grapefruit seed extracts, colloidal silver, oregano, thyme, rosemary oil, garlic is pivotal in order to select a tailored antibiotic therapy to effectively treat the patient.

Sepsis can result from an infection anywhere in the body, such as pneumonia, influenza, or urinary tract infections. Moreover, sepsis can be caused by food-borne infections, for example by ingesting contaminated food.

Microbial pollution is an environmental problem and during the past decades the microbial pollution has been increasing and is considered as important issue in food security. Microbial pollution is a serious issue because it can lead to a wide range of health problems. A great number of foodborne diseases and outbreaks are reported in which contamination of fresh produce and animal products occurs from polluted sources with pathogenic bacteria, viruses and protozoa. Besides diseases and death, the consumption of pathogen contaminated foods also creates economic impact that can be quite devastating on the consumers, a nation, food dealers and food companies. Pathogenic bacteria, viruses and protozoa could be introduced to the foods of both animal and non-animal products during: (1) primary production (in the farm where plants are grown or animals are raised for food; (2) at harvest and slaughter of food produce and food animals respectively; (3) transportation; (4) food processing; (5) storage; (6) distribution and (7) preparation and serving (Bintis et al, 2018, AIMS Microbiol. 4(3), 377-396). The most common sources of environmental pollution with microorganisms occur in the primary production. Therefore, easy and fast measurements to control the contaminations at the earlier stages of the food chain are required.

The majority of clinical microbiology laboratories still rely on conventional methods, such as culture-based techniques, microscopy, Gram-staining and biochemical tests, for detecting microbial pathogens in samples. Traditionally, microbiological culturing is performed using general purpose agar-based media such as blood agar, that will support the growth of a wide range of pathogens, differential media that target differences in the metabolic activity of microbes utilizing biochemical indicator systems (e.g. the incorporation of sugar in addition to a pH indicator), or selective media that incorporate specific antimicrobials (Varadi et al., 2017, Chem. Soc. Rev., 46, 4818-4832). Although the conventional methods are well adapted, rather inexpensive, and provide valuable information about a wide range of pathogens, they are laborious, time-consuming, and often not sufficient to detect very small amounts of microbes occurring in fluid or due to antibiotic treatment. Other well-established methods include immunological techniques that rely on binding of antibodies to specific antigens of target microbes (e.g. enzyme-linked immunosorbent assays (ELISA) or serological assays), nucleic acid-based techniques that include variations of hybridization, polymerase chain reaction, sequencing, DNA/RNA microarrays, and in particular Matrix assisted laser desorption/ionization-time of flight mass spectrometry (MALDI-TOF MS) which has revolutionized the identification of bacterial pathogens since its introduction in 2010 and which is now the method of choice in most advanced clinical laboratories. In MALDI-TOF MS, the sample is treated with a matrix, which absorbs energy from a laser resulting in rapid heating, vaporization, and ionization of the analytes; the ions are then separated on the basis of the time they take to reach the detector, as all ions of the same charge are given the same kinetic energy (Varadi et al., 2017, Chem. Soc. Rev., 46, 4818-4832). However, despite of the high success rate of these methods, they are still very time consuming and require extensive sample preparation. Further limitations include low specificity and sensitivity, necessity of pre-cultivation of the microorganisms, requirement of labelling and high background noise, which delay or impact a reliable characterization of bacterial pathogens (Sousa et al., 2013, Science, Technology and Education, Vol. 3, 1429-1438). However, during microbial infections, in particular sepsis, the initial hours (up to 3 hours) are considered essential for a patient's prognosis and the overall mortality rate.

Hence, there is a need for an improved in vitro analysis methodology, which allows for a rapid and reliable detection and evaluation of microbial pathogens in liquid samples.

### OBJECTS AND SUMMARY OF THE INVENTION

The present invention addresses these needs and provides in one aspect an in vitro method for analysing a liquid sample as to the presence, identity and properties of microbes comprising: a) isolating microbes from a liquid sample; b) analysing said microbes spectroscopically by means of spontaneous Raman spectroscopy; and c) determining antibiotic susceptibility of said microbes spectroscopically by means of spontaneous Raman spectroscopy. This approach is highly advantageous since obtaining and analysing liquid samples in vitro by means of spontaneous Raman spectroscopy is extremely fast, minimally invasive and much less costly to perform when compared to tissue biopsy procedures. In particular, the extremely time-consuming cultivation and staining of microbes can largely be avoided, thus allowing for a direct microbiological assessment within a short period of time well below the critical 3 hours time limit for e.g. sepsis diagnosis. In addition, it is easily accessible, may allow for stratification and real-time monitoring of therapies, and can be easily repeated. Furthermore, the methodology does not require the presence of marker genes or staining steps since it is based on the reaction of the liquid sample components on stimulation with laser radiation and the subsequent recording of spontaneous Raman spectra.

In one embodiment, the isolation in step a) is performed by centrifugation or filtration or said liquid sample.

In another embodiment, said filtration is performed in a chip designed to size-exclude components within the liquid sample which are larger than microbes.

In yet another embodiment, the microbes are enriched in a micro-chamber of the chip.

In a further embodiment, said chip is a part of a microfluidic system.

In a preferred embodiment, the above method additionally comprises as step a-(i) a quantification of the isolated microbes.

In another embodiment, the quantification is performed by means of image analysis of isolated microbes within a channel of the chip.

According to a preferred embodiment, steps b) and c) comprise recording at least one Raman spectrum by means of Raman spectroscopy of an isolated microbe.

In yet another embodiment, the analysis of step b) and the determination of step c) comprises collecting and arresting a microbe in an optical trap in order to record a Raman spectrum.

In a further embodiment, said optical trapping forces are produced simultaneously by means of an excitation beam of a Raman spectroscopy system.

In a further embodiment, the analysis of step b) and the determination of step c) comprises collecting, slowing down movement and arresting a microbe in a decelerating material such as a fibrous gel, a hydrogel or collagen gel in order to record a Raman spectrum.

According to a preferred embodiment, step b) comprises a comparison of the Raman spectrum obtained from the microbe isolated in step a) with a reference spectrum, preferably derived from a database, thereby determining the identity of said microbe.

In a further embodiment, said determination of antibiotic susceptibility of the microbes in step c) comprises obtaining a Raman spectrum for a microbe prior and subsequent to the exposure of the microbe to the antibiotic.

In one embodiment, said microbe is exposed to the antibiotic for about 0.5 to 30 minutes.

In another embodiment, said microbe is exposed to a single antibiotic or to a combination of at least two different antibiotics simultaneously or sequentially, preferably in the form of a gradient of said antibiotic or said combination of antibiotics.

In yet another embodiment, said antibiotic is provided to the microbe at one or more micro-chambers within said chip.

According to one embodiment, the method comprises conducting a statistical evaluation of the at least one Raman spectrum.

In a further embodiment, the method comprises a principal component analysis and/or cluster analysis of the at least one Raman spectrum.

In a further embodiment, the method comprises a principal component analysis and/or cluster analysis and/or a linear discriminant analysis (LDA) of the at least one Raman spectrum.

In a preferred embodiment, the method comprises a spectral analysis of the Raman spectrum.

In a further preferred embodiment, the method comprises statistical evaluation and judgment on the basis of artificial intelligence and/or machine learning algorithms for complex matrix data evaluation.

In one embodiment, the method is performed computer-based, preferably automatically or semi-automatically.

In another embodiment, the liquid sample is (i) a body fluid sample, preferably blood or banked blood, bile, urine, saliva, pleural fluid, ascites, cerebrospinal fluid, amniotic fluid or bronchoalveolar lavage fluid sample, or (ii) an environmental sample, preferably a food sample or a drinking sample.

In a further embodiment the method is for the detection of sepsis in a subject. It is particularly preferred that the method is for the detection of sepsis and antibiotic susceptibility of the sepsis' causative agents.

A further aspect of the present invention relates to a device for analyzing a liquid sample as to the presence, identity and properties of microbes, wherein the device comprises as a first unit (i) a chip comprising a filtering unit; or a chip comprising a filtering unit and an antibiotics exposure unit capable of determining the susceptibility of microbes to an antibiotic; as a second unit (ii) a Raman spectroscopy system; and as a third unit (iii) an evaluation module which is coupled to the Raman spectroscopy system.

In one embodiment, said device comprises as a forth unit (iv) a microfluidic component for semi-automated measurement and/or transporting and/or separating said liquid sample or microbes, which is coupled to the Raman spectroscopy system.

In another embodiment, said device further comprises an integrated optical trapping module.

In a further embodiment, the filtering unit of the chip is designed to size-exclude components within the liquid sample which are larger than microbes, thereby isolating said microbes.

In yet another embodiment, the antibiotics exposure unit of the chip comprises one or more micro-chambers comprising an antibiotic or a combination of antibiotics, wherein said antibiotic or said combination of antibiotics is preferably lyophilized.

In a preferred embodiment, said evaluation module is designed to perform principal component analysis and/or a normalization on specific band and/or cluster analysis and/or LDA analysis.

In a further embodiment, said evaluation module is configured to analyse isolated microbes by comparing the Raman spectrum obtained from an isolated microbe with a reference spectrum, preferably derived from a database.

In a further embodiment, the evaluation module is designed to perform a statistical evaluation and judgment on the basis of artificial intelligence and/or machine learning algorithms for complex matrix data evaluation. In one embodiment, the device is configured to perform the method described above.

Yet another aspect of the present invention refers to a system comprising the aforementioned device and a module comprising a database comprising reference values of Raman spectra obtained from microbes.

In a further aspect the present invention relates to the use of a method as defined herein above, of the device as described ab above, or of a system as described above for the detection of sepsis in subject.

In a preferred embodiment, the invention relates to said use for the detection of sepsis and antibiotic susceptibility of the sepsis' causative agents.

In a further embodiment, the invention relates to the method as defined herein above, to the device as defined herein above, to the system as defined herein above or the use as defined herein above, wherein said microbe is a bacterium, a unicellular fungus or a protist. It is particularly preferred that said bacterium is of the genus Acinetobacter, Klebsiella, Pseudomonas, Escherichia, Enterobacter, Enterococcus, Staphylococcus, or Streptococcus, and that the unicellular fungus is of the genus Candida.

### BRIEF DESCRIPTION OF THE DRAWINGS

FIG. 1 shows microscopy pictures of the morphology of four bacteria samples: (A) Bacillus cereus, (B) Micrococcus luteus, (C) Escherichia coli and (D) Pseudomonas aeruginosa.

FIG. 2 shows steps of a trapping procedure (from left to right), wherein (i) a bacterial cell is targeted; (ii) the Raman excitation laser is switched on (bright spot), simultaneously a bacterium is trapped; (iii) by changing the microscope focus, the trapped bacterium is lifted in the z direction; (iv) moving the microscope stage in x/y direction causes the bacterium to move accordingly; (v) finally, switching off the laser releases the bacterium which moves away unaffectedly.

FIG. 3 shows overlay plots of processed Raman spectra of four bacteria samples (Bacillus cereus, Micrococcus luteus, Escherichia coli and Pseudomonas aeruginosa). The bacteria correspond to those shown in FIG. 1. Ten single bacteria of each sample were trapped and measured. Each single thin line represents one Raman spectrum of one single bacterium.

FIG. 4 depicts an overlay of the mean spectra of the four bacteria samples depicted in FIG. 3.

FIG. 5 shows principal component analysis (PCA) score plots of all the measured data from the four bacteria samples depicted in FIGS. 3 and 4.

FIG. 6 shows bar plots of the second and third two principal component of all measured data.

FIG. 7 shows loading plots of the first two principal component of all measured data. Loading and bar plots reveal the magnitude of spectral variations of each principal component.

FIG. 8 shows an example of a channel slide and membrane slide with borosilicate bottom.

FIG. 9 provides a schematic overview of the architecture of the microfluidic chip with areas of antibiotic testing (dark oval areas at right hand side).

FIG. 10 shows details of a bacterial identification approach for sepsis detection according to embodiments of the present invention.

FIG. 11 depicts an example of a chip comprising two chambers, A and B, and a 1 μm pore-size filter according to an embodiment of the present invention.

FIG. 12 shows a further example of a chip comprising two chambers and a filter according to an embodiment of the present invention.

FIG. 13 shows leaves of fresh and 7 days old Iceberg salad, which where immersed in PBS buffer and mixed for 5 min (see FIG. 13 left). 50 μm were taken and pipetted into microchannel chips (see FIG. 13 right). Raman measurements of single bacteria were performed.

FIG. 14 shows a microscopic view of microbes from fresh (see FIG. 14 left) and 7 days old lettuce (see FIG. 14 right). The amount of microbes is much higher in old lettuce. In addition, there are different morphologies varying from small round bacteria up to rod like fungi.

FIG. 15 depicts Raman spectra of different lettuce species. The figure reveals clear differences in the spectral patterns.

FIG. 16 depicts a mean Raman spectra overlay. The figure shows the differences between the microbes.

FIG. 17 depicts a score plot after Principal component analysis (PCA) of the data shown in FIG. 15 or 16. This figure demonstrates the differences (in this preliminary test only 5 cells per samples were measured).

FIG. 18 depicts the Raman spectra of bacteria treated with ofloxacin (shown in grey) the same bacteria without any treatment (control—shown in black), Raman spectra changes are clearly observed.

FIG. 19 depicts the principal component analysis (PCA) of the Raman data: in (a) the score plot revealing different scattering patterns between control bacteria (black dots) and bacteria treated with ofloxacin are shown (star-like dots); in (b) the loadings plot illustrating the difference of the Raman bands that was used for classifications are shown.

FIG. 20 depicts Raman raw spectra of erythrocytes vs. erythrocytes after 3 hers of incubation.

FIG. 21 shows mean Raman spectra of erythrocytes vs. erythrocytes after 3 hours of incubation.

FIG. 22 shows a score plot after Principal component analysis (PCA) of the data shown in FIG. 20 or 21.

FIG. 23 depicts the PC scores illustrating the difference of the Raman bands that were used for classifications.

FIG. 24 shows Raman raw spectra data of erythrocytes and bacteria.

FIG. 25 shows that erythrocytes and bacteria produce different mean Raman spectra.

FIG. 26 shows a score plot after Principal component analysis (PCA) of the data shown in FIG. 24 or 25.

FIG. 27 depicts the PC scores illustrating the difference of the Raman bands that were used for classifications.

FIG. 28 provides an overview of the automated data and spectra processing steps implemented in an embodiment of the present invention.

FIG. 29 depicts the detection of airborne microorganisms. In FIG. 29A mean spectra of different bacteria and fungi are shown. FIG. 29B shows the bacteria and fungi colonies on agar plate which were used for the measurement shown in FIG. 29A.

FIG. 30 shows the spectra of Staphylococcus aureus and Staphylococcus epidermis. Main peaks within the mean spectra represent carotinoids

FIG. 31 shows the results of the principal component analysis of Raman spectra as depicted in FIG. 30, as well as the agar plate the bacteria are derived from and microscopic images of the bacteria.

FIG. 32 shows results of the score plots of the principal component analysis of Raman spectra of different strains of Pseudomonas (P-50BA, P-52BA, P-80BA) and Staphylococcus (S-404 and S-407). The different bacteria species are assembling in clearly distinct clusters.

FIG. 33 shows mean spectra of EHEC bacteria and of Staphylococcus aureus (A), as well as score plots of the corresponding principal component analysis (B). The results demonstrate a clear discrimination between the tested bacteria.

FIGS. 34 and 35 show mean spectra of EHEC 52371 and EHEC 5756 (A), as well as score plots of the corresponding principal component analysis (B). The results demonstrate a clear discrimination between the tested EHEC strains.

FIG. 36 provides an overview of mean spectra of Escherichia coli 11701 and Escherichia coli 15787 and Escherichia alberti 18145, which differ in several peaks. Discriminating peaks are marked with an arrow and described including wavenumber and corresponding biomolecules such as tryptophane, tyrosin or cytosins.

FIG. 37 shows score plots of the corresponding principal component analysis of the mean spectra depicted in FIG. 36. Shown are three clearly distinct clusters with only minimal overlap.

FIG. 38 shows mean spectra of Escherichia coli strain 15787 in normal vs hunger medium. The spectra clearly differ in several peaks.

FIG. 39 shows score plots of the corresponding principal component analysis of the mean spectra depicted in FIG. 38. Shown are two clearly distinct clusters.

FIG. 40 shows mean spectra of Listeria bacteria (dead or alive) (A), as well as score plots of the corresponding principal component analysis (B). The obtained mean spectra of the dead/live bacteria samples have peaks that clearly differ from each other and the corresponding score plot of the two samples depict clearly distinct clusters.

### DETAILED DESCRIPTION OF THE EMBODIMENTS

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use, there is no time or time interval coherence between the steps, i.e. the steps may be carried out simultaneously or there may be time intervals of seconds, minutes, hours, days, weeks, etc. between such steps, unless otherwise indicated.

It is to be understood that this invention is not limited to the particular methodology, protocols, reagents, etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect an in vitro method for analysing a liquid sample as to the presence, identity and properties of microbes comprising: a) isolating microbes from the liquid sample; b) analysing said microbes spectroscopically by means of spontaneous Raman spectroscopy; and c) determining antibiotic susceptibility of said microbes spectroscopically by means of spontaneous Raman spectroscopy. In certain embodiments, step c) may be optional, i.e. the method does not comprise the determination of an antibiotic susceptibility. Also envisaged are correspondingly designed devices as mentioned below.

The present invention further contemplates an in vitro method for determining a microbial contamination or a microbial infection in a liquid sample, preferably a liquid from a subject or from food, blood products or tools, by using Raman spectroscopy comprising (i) isolating microbes from the liquid sample; (ii) analysing the microbes spectroscopically by means of spontaneous Raman spectroscopy; and (iii) obtaining a Raman spectrum for said microbes.

As used herein, the term “liquid sample” refers to a liquid material obtained via suitable methods from one or more biological organisms or comprising one or more biological organisms, or processed after having been obtained. The liquid sample may further be material obtained from contexts or environments in which biological organisms are present, or processed variants thereof. Typically, the liquid sample is an aqueous sample. In preferred embodiments, it may comprise a bio-organic fluid obtained from the body of a mammal that is taken for analysis, testing, quality control, or investigation purposes. In a preferred embodiment, said liquid sample may be blood, blood components or banked blood, bile, urine, saliva, nasal fluid, ear fluid sweat, sputum, semen, breast fluid, milk, colostrum, pleural fluid, ascites, cerebrospinal fluid, amniotic fluid or bronchoalveolar lavage fluid, gastric fluid, aqueous humor, vitreous humor, gastrointestinal fluid, exudate, transudate, pleural fluid, pericardial fluid, upper airway fluid, peritoneal fluid, liquid stool, fluid harvested from a site of an immune response, or fluid harvested from a pooled collection site. Furthermore, the liquid sample may contain a tissue extract derived from body tissues, e.g. tissues obtained via biopsy or resections, preferably from a eukaryotic organism, more preferably from a mammalian organism, even more preferably from a human being. The biopsy material may be derived, for example, from all suitable organs, e.g. the lung, the muscle, brain, liver, skin, pancreas, stomach, heart, stomach, intestine etc., a nucleated cell sample, a fluid associated with a mucosal surface, or skin. In order to be extracted, the biopsy material is typically homogenized and/or resuspended in a suitable buffer solution as known to the skilled person. Such samples may, in specific embodiments, be pre-processed e.g. by enrichment steps and/or dilution steps etc.

In specific embodiments, the “liquid sample” may also encompass a non-bioorganic fluid that is, for example, taken for analysis or quality control purposes, including but not limited to vaccines, liquid pharmaceutical formulations, medical solutions and drops, and the like.

In further specific embodiments, the “liquid sample” may encompass a fluid obtained from food, for example vegetables such as cabbage, salad, fruits, etc. The “liquid sample” may also be derived from drinks or drinkings in any form, water, beverages such as fruit juice, tea, coffee, milk, etc. The liquid sample may also be derived from solutions of medicinal products such as cell therapeutics, blood products, tissue grafts, etc., or from liquids obtained from medical devices such as scalpels, tubes, bottles, flasks, etc.

The term “microbe” or “microbes” refers to a microorganism, which typically exits in a single-celled form or may form clusters or colonies, which do not show internal cellular differentiations. Within the context of the present invention, the microbes may be, for example, archaea, bacteria, eukaryotic single cell organisms such as protists or unicellular fungi. The term “bacterium” or “bacteria” refers to any bacterium known to the skilled person. The term, in particular, relates to pathogenic or contaminating bacteria in the context of health and hygiene, as well as commensal bacteria of mammals, in particular the human being, or any other type of bacterium present in the environment of human beings. Further information can be derived, for example, from suitable database resources such as the National Microbial Pathogen Data Resource (NMPDR) which is accessible at http://www.nmpdr.org; Microbiomes Online which is accessible at http://www.microbesonline.org. In preferred embodiments, the bacterium is a bacterium associated with sepsis. Examples of such bacteria include Streptococcus pneumoniae, Haemophilus influenzae, Staphylococcus aureus, in particular MRSA, Escherichia coli, Salmonella spp. and Neisseria meningitidis. “Archaea” are single celled microorganisms, which typically lack cell nuclei and comprise a genetic composition which is different from the bacterial genotype. Furthermore, the archaea rely on lipids in their cell membrane and are not capable of forming endospores. Examples of archaea Methanococci, Eurythermea, Neobacteria, or Diapherotrites. “Eukaryotic unicellular fungi” include, for example, yeasts such as Kluyveromyces, Pichia, Saccharomyces or Candida. The term “protist” as used herein relates to flagellata, ciliphora or sporozoa. Examples of protists are the genus, Plasmodium, Trypanosoma, Entamoeba, Balantidium, Amoeba, Syringammina, Bodo, or Nocto.

The “identity” of microbes refers to a characterization of the microbe with respect to its taxonomic status. It is preferred that the identity of the microbe also includes information on the pathologic status of the microbe. The identity of the microbe may be determined on the level of sub-species or variety, species, genus, family or order. For example, the affiliation of a microbe to a specific species, a specific sub-species, a specific family or a specific order may be achieved when performing the present invention. In further specific embodiments, the determination of identity may include the differentiation of two or more microbe sub-species, species, genus, family or orders when present in a liquid sample. The taxonomic basis for the characterization of the microbe with respect to its identity may follow the skilled person's knowledge about current taxonomic definitions, e.g. fueled by morphologic, biochemical or genetic properties of an organism. Non-limiting examples of microbes which can be identified according to the present invention include bacteria such as Achromobacter, Acinetobacter, Brucella, Cyanobacterium, Pseudomonas, Helicobacter, Escherichia, Salmonella, Shigella, Enterobacter, Klebsiella, Serratia, Proteus, Oligoflexia, Campylobacter, Haemophilus, Listeria, Morganella, Vibrio, Shigella, Spirochaeta, Treponema, Wolbachia, Yersinia, Stenotrophomonas, Brevundimonas, Ralstonia, Fusobacterium, Prevotella, Branhamella, Neisseria, Burkholderia, Citrobacter, Hafnia, Edwardsiella, Aeromonas, Moraxella, Pasteurella, Providencia, Staphylococcus, Streptococcus, Legionella, more preferably Acinetobacter baumannii, Bacteroides fragilis, Bordetella japonica, Devosia pacifica, Enterobacter cloacae, Flavobacterium akiainvivens, Gluconacetobacter diazotrophicus, Haemophilus haemolyticus, Hemophilus influenza, Klebsiella pneumoniae, Legionella pneumophila, Moraxella bovis, Neisseria gonorrhoeae, Proteus mirabilis, Pseudomonas aeruginosa, Rickettsia rickettsii, Salmonella enterica, Serratia marcescens, Vibrio cholera, Staphylococcus aureus, Staphylococcus epidermidis, Streptococcus pneumoniae, Haemophilus influenzae, Escherichia coli, EHEC, Salmonella spp. and Neisseria meningitides, or eukaryotic unicellular fungi such as Kluyveromyces, Pichia, Saccharomyces or Candida, e.g. Candida albicans. The term “protist” as used herein relates to flagellata, ciliphora or sporozoa. Examples of protists are Plasmodium, Trypanosoma, Entamoeba, Balantidium, Amoeba, Syringammina, Bodo, and Nocto.

The term “microbial contamination” or “microbial infection” as used herein relates to the presence of a microbe as defined herein above in a sample, preferably a liquid sample. The microbe may typically be present in a certain amount to be detectable, or can be detected after suitable culturing activities. Furthermore, one, two or more different microbes or groups of microbes may contribute to a microbial contamination or microbial infection. The contamination may, for example, be detectable in the context of food, blood products (e.g. blood), technical instruments or devices or the like, whereas an infection may be detectable in the context of samples derived from a subject.

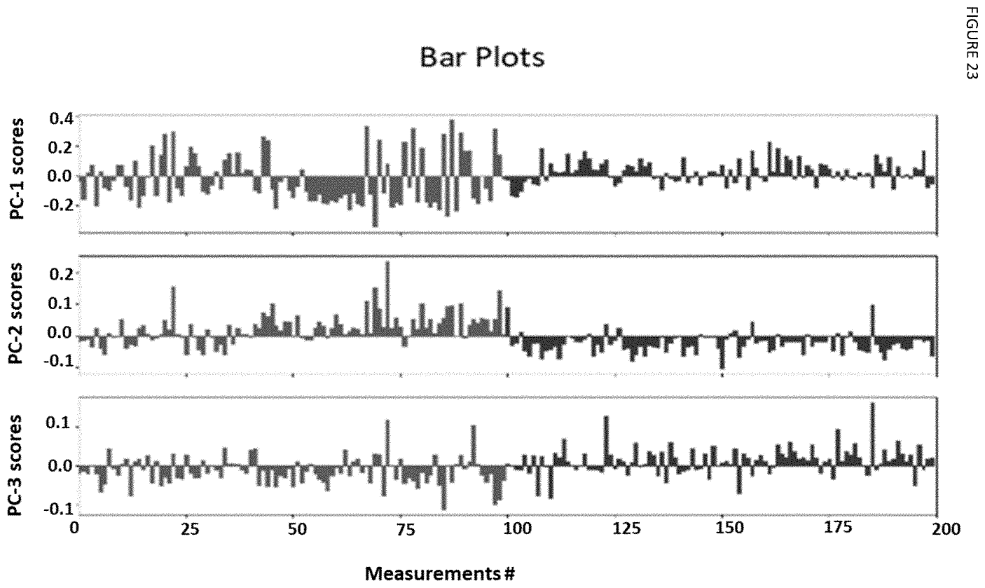

As can be derived from FIG. 23 and Example 4 an in vitro method for determining a microbial contamination or a microbial infection in a liquid sample by using Raman spectroscopy comprising (i) isolating microbes from the liquid sample; (ii) analysing the microbes spectroscopically by means of spontaneous Raman spectroscopy; and (iii) obtaining a Raman spectrum for said microbes is advantageously capable of distinguishing between cells in the presence of microbes and in the absence of microbes. The present invention accordingly envisages a method for determining a microbial contamination or a microbial infection in a liquid sample by analysing the cell spectroscopically by means of spontaneous Raman spectroscopy; obtaining a Raman spectrum for said cells; and comparing said Raman spectrum to a control spectrum, preferably of cells of the same type which have not been in contact with a microbe. The present invention further relates to a method for analysing spectroscopically by means of spontaneous Raman spectroscopy a microbe or a cell being in direct or indirect contact with a microbe in a liquid sample. The method comprises steps as defined herein. It is preferred that the cell is an erythrocyte.

The term “properties of microbes” refers to an inherent or acquired characteristic of a microbe. For example, the term may relate to a pathological status or quality of a microbe, to a genetic or biochemical property, or to reactivity behaviour, preferably it relates to an antibiotic susceptibility. The term “antibiotic susceptibility” as used herein relates to the sensitivity of microbes to one or more chemical compounds or composition, in particular to one or more antibiotic compounds or antibiotic compositions, as well as to one or more bactericidal compounds or bactericidal compositions. Antibiotics are antimicrobial substances active against microbes, in particular bacteria, and thus, are widely used in the treatment and prevention of microbial infections. Antibiotics are commonly classified based on their mechanism of action, chemical structure, or spectrum of activity. For example, they may target microbial functions, growth processes, cell wall or cell membrane, or interfere with essential enzymes. Further categorization of antibiotics refers to narrow-spectrum antibiotics, which target specific types of cells, e.g. bacteria, such as gram-negative or gram-positive, or broad-spectrum antibiotics, which affect a wide range of microbes, in particular bacteria. Typical examples of antibiotic classes include but are not limited to penicillins (e.g. amoxicillin, ampicillin, oxacillin, dicloxacillin, etc.), tetracyclines (e.g. demeclocycline, doxycycline, eravacycline, minocycline, omadacycline, etc.), cephalosporins (e.g. cefaclor, cefotaxime, ceftazidime, cefuroxime, etc.), quinolones (e.g. ciprofloxacin, levofloxacin, moxifloxacin, etc.), lincomycins (e.g. clindamycin, lincomycin, etc.), sulphonamides (e.g. sulfamethoxazole and trimethoprim, sulfasalazine etc.), glycopeptide antibiotics (e.g. dalbavancin, ortavancin, telavancin, vancomycin, etc.), aminoglycosides (e.g. gentamicin, tobramycin, amikacin, etc.) and carbapenems (e.g. imipenem and cilastatin, merpoenem, dorpenem, ertapenem, etc.). Further information on antibiotics and their function, as well as resistance mechanisms, can be derived from suitable literature or database resources such as Comprehensive Antibiotic Resistance Database which is accessible at https://card.mcmaster.ca.

The susceptibility to antibiotics may vary between and within a species of microbes, as well as with the concentration of the antibiotic. Some microbes are resistant to one antibiotic, other microbes may have developed a resistance to more than one antibiotic, i.e. are multiple resistant and thus are difficult to treat, e.g. by requiring alternative medications or higher doses of antimicrobials. Thus, the present invention envisages a method of determining the susceptibility of microbes to an antibiotic or a combination of antibiotics.

In a first step of the method according to the present invention, the microbes are isolated from a liquid sample as described herein above. As used herein, the term “isolation” or “isolating” refers to a process of removing or otherwise setting apart microbes from their original liquid sample and/or from other components in said liquid sample. The term may further relate to a process of concentration of microbes within the original liquid sample, whereby significant amounts of the original liquid sample are removed, while microbes are not removed. The term may, in certain embodiments, further include an at least partial purification of microbes from the liquid sample, or from any non-microbes or non-microbial component within the sample. For example, microbes may be isolated from non-microbes or non-microbial components that may otherwise interfere with characterization and/or identification of the bacteria. Typical examples of such non-microbial components include non-microbial cells such as blood cells and/or other tissue cells, and/or any components or fragments thereof. The isolation may, in certain embodiments, further envisage an isolation of different classes of microbes, e.g. allow for an isolation of bacteria from non-bacteria, or allow for an isolation of prokaryotic cells from eukaryotic cells, or allow for an isolation of archaea from other microbes etc. The isolation may, in certain embodiments, result in the provision of a collection or layer or accumulation of microbes or sub-classes thereof as defined herein, wherein the microbes are more concentrated than in the original liquid sample. In certain embodiments, said accumulation is present within the context of the original liquid sample, or outside of the context of said original liquid sample. Such a concentrated layer or accumulation of microbes may range from a closely packed dense clump of microbes to a diffuse layer of microbes.

In a preferred embodiment the isolation of microbes is performed by centrifugation or filtration. The term “centrifugation” refers to the rotation of a sample to generate a centrifugal force for separating microbes from other ingredients of the liquid sample according to their size, shape, or density. As used herein, centrifugation involves the separation of microbes from other ingredients of the liquid sample by compacting of microbes into a microbe rich zone or a pellet by applying centrifugal forces on a liquid sample containing microbes by using suitable centrifugal forces. For example, the centrifugal forces may range from about 500 to 12,000×g. Furthermore, the effect of the centrifugation may be controlled by the use of suitable centrifugation times. For example, the centrifugation may be performed for about 10 sec to 5 min. In preferred embodiments, the isolation by centrifugation may be implemented by using a high-density cushion. For example, the microbes may be sandwiched between two layers, e.g. microbes collected on top of a high-density cushion after centrifugation. Alternatively, the microbes may be collected on a solid surface, e.g. as a layer. Examples of suitable surfaces include solid substrates, glass surfaces or a filter membrane. The process of centrifugation may further be improved by using a density gradient centrifugation medium. Examples of suitable density gradient centrifugation media include OptiPrep, Percoll, Ficoll 400, Ficoll-Paque and Ficoll-Hypaque PLUS.

It is particularly preferred that the microbes are isolated via filtration. The term “filtration” as used herein refers to a separation process based upon the size difference between the suspended particles, e.g. microbes or biological particles in the sample such as non-microbial cells or cell components/fragments, and the size of the passageways, i.e. pores, present in or on the filter. The filtration is hence designed to size-exclude components within the liquid sample which are larger than microbes, thereby allowing for a separation and, in consequence, isolation of microbes.

A filter may, in typical embodiments, be composed of a filter membrane. According to the present invention a “filter membrane” may be a membrane material comprising single-layer, woven nylon meshes, or being composed of cellulose acetate, polyethylether, nylon, glass fiber or polytetrafluorethylene. Further envisaged are hydrogel, collagen-gel or alike fibrous materials. The membrane may have pores of a range of suitable maximum diameters so as to prevent or allow the passage of microbes. The filter membrane may, in preferred embodiments, be filter membrane for microfiltration (pore size of >0.1 μm) or ultrafiltration (pore size of 100-2 nm). Alternatively, the filtration function may also be performed by non-classic filters such as silicon nitride layers. Such layers are envisaged to comprise micro-holes, e.g. in the range of 1.5 to 3 μm (diameter) or of about 0.22 μm to 0.45 μm (diameter). It is further envisaged that a filter of biologic origin be used. Suitable examples include filters comprising agarose, hydrogel and/or collagen material.

In this context, microbes may be isolated from larger components of the samples, e.g. blood cells or other non-microbial cells or particles, by filtration through a filter membrane having pore sizes of about 1.5 to 3 μm (diameter). By using such a pore size microbes, which are assumed to have an average diameter of about 0.6 to 1.0 μm will not be retained by the filter membrane, thus pass said membrane and can be collected together with the liquid portion of the sample, whereas larger particles will be retained at the pores or holes of the membrane.

Alternatively or additionally, a second filtration process may be used to concentrate microbes and/or to separate microbes from liquid components of the sample. Accordingly, a filtration through a filter membrane having pore sizes of about 0.22 μm to 0.45 μm (diameter) may be performed. By using such a pore size microbes, which are assumed to have an average diameter of about 0.6 to 1.0 μm will be retained by the filter membrane and can thus be collected on the pores of the filter membrane, whereas the liquid portion of the sample passes said membrane. Accordingly, microbes may be isolated and/or separated from liquid components of the sample.

In a preferred embodiment, isolation of microbes by filtration is performed in a chip designed to size-exclude components within the liquid sample which are larger than microbes. The term “chip” relates to a silicon unit or silicon-derivative unit, which is capable of separating microbes from other components present in a sample as defined above, of isolating microbes and of presenting microbes to subsequent analysis steps, in particular spectroscopic analyses by means of spontaneous Raman spectroscopy as described herein. In certain embodiments, the chip is capable of retaining microbes in suitable chambers and allows for transport, cultivation and analysis of microbes. The cultivation and analysis functions may, in preferred embodiments, be performed in specific micro-chambers or zones of the chip, which are connected to channel- or passage-structures, e.g. in the form of micro-channels. The transport function may be implemented via the micro-channel(s) and/or main-channels, which may split or open into several micro-channels which in turn end in micro-chambers. Transport of microbes into micro-chambers as defined herein may be implemented in various suitable ways. For example, microfluidic techniques as described in more detail below can be used to transport the microbes into and out of the chamber. Also the transport of medium, ingredients, antibiotics etc. may be performed with microfluidic elements such as laminar flows, capillary forces etc. Alternatively, the transport of microbes, as well as their arrest at specific locations, e.g. in a micro-chamber, may be performed with electromagnetic forces, preferably with optical tweezers as defined herein below. In further embodiments, electromagnetic gradients between electric poles, i.e. plus and minus, may be sued. Further envisaged are induced electrical fields, or centrifugal forces which are applied to the microbes. In a preferred embodiment, a correspondingly designed fluidic channel may be have the form of a spiral with chambers located at the outside, designed to receive the microbes upon application of the mentioned forces, e.g. centrifugal forces.

A chip may comprise an inlet, e.g. for injection of liquid samples, which may be injected into the inlet via a syringe or the like, as well as a multitude of micro-chambers and corresponding micro-channels, e.g. between 2 to 1000 separate micro-chambers and corresponding micro-channels, which may be arranged in any suitable manner to allow for a transport, cultivation and analysis of microbes. For example, the micro-chambers may be located in a star-like manner around a central channel structure. Alternatively, the micro-chambers may be arranged at both sides of street-like oriented main-channel. Also envisaged is a ring-like or spiral-like main-channel with micro-chambers at both sides. These channels preferably are used in embodiments in which centrifugal forces are applied. It is preferred that the micro-chambers are used for the enrichment of microbes, e.g. via transport processes or arresting procedures as described herein. In specific embodiments, some of the micro-chambers are designed for cultivation of microbes, e.g. by comprising cultivation medium, or by having a connection to a channel transporting cultivation medium to the microbes.

In further embodiments, the chip comprises an antibiotics exposure unit capable of and designed for determining the susceptibility of microbes, in particular bacteria to an antibiotic. The antibiotics exposure unit is designed as a micro-chamber which may be located in a specific part of the chip. In an embodiment, the antibiotics exposure unit of the chip may comprises one or more micro-chambers comprising an antibiotic or a combination of antibiotics. Accordingly, the micro-chamber comprises a suitable amount of an antibiotic or a combination of antibiotics, e.g. one of the antibiotics as mentioned above, or is connected to a reservoir or channel transporting the antibiotic to the micro-chamber. In a preferred embodiment, said antibiotic or said combination of antibiotics is lyophilized. The term “lyophilized” refers to the state of an antibiotic, wherein it underwent a freeze-drying process to remove water from the antibiotic after it is frozen and placed under a vacuum. It is envisaged herein that said lyophilized antibiotic is activated upon contact with a liquid, which is, for example, provided to the chambers via the inlet of the chip. When the isolated microbes come into contact with the activated antibiotics, the susceptibility to said antibiotics may be determined by recording and comparing Raman spectra prior and subsequent to the exposure to the antibiotics as described herein. In further embodiments, the determination of a susceptibility to antibiotics may be performed with the support of Raman spectra databases as described herein. A comparison with data sets in the database may be performed, for example, during or after the determination. The microbes to be analyzed may be derived directly from samples, or may have been prepared, processed or cultured before determination, or have been enriched before determination.

In a specific embodiment the chip comprises μm sized channels. Alternatively, the chip comprises μm sized channels with an integrated filtering unit. In a further alternative embodiment the chip comprises μm sized channels with an integrated filtering unit and an antibiotics exposure unit capable of determining the susceptibility of microbes to an antibiotic.

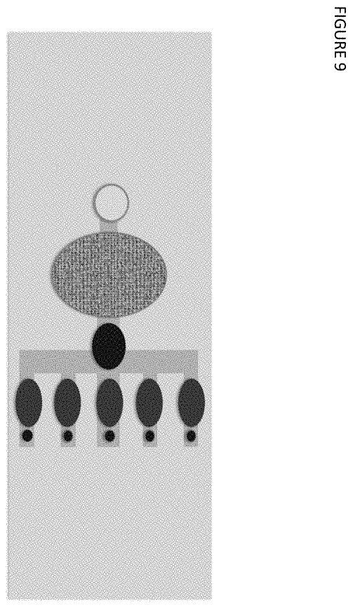

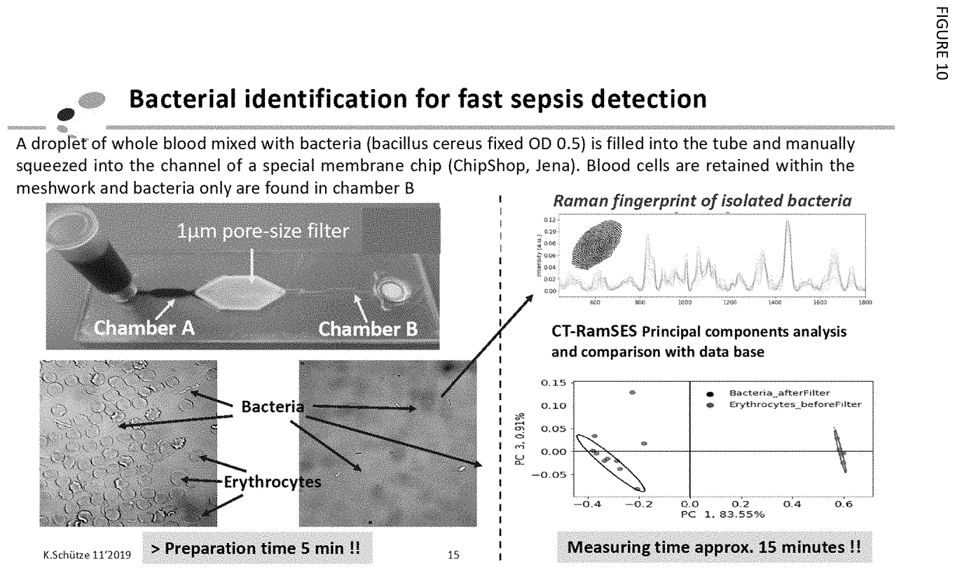

An exemplary version of the chip is shown in FIG. 9.

The chambers and channels of the chip may have any suitable size, preferably in the μm range. Its is accordingly envisaged in a preferred embodiment to provide micro-chambers with a height of about 50 to 200 μm and diameter of about 50 to 500 μm. The filter area could cover an area of about 1 mm² up to 1 cm². The main channel may preferably have a width of about 50 to 300 μm, a height of about 50 to 200 μm and a length of about 100 μm to 1 cm or more, depending on the number of micro-chambers. Side channels connecting the main channel and the micro-chamber may preferably have a height of about 50 to 200 μm and a length of about 50 to 80 μm with a width of about 10 to 30 μm.

The chip may be composed of any suitable material. It is preferred that the material is at least partially translucent and allows for spectroscopic analyses by means of spontaneous Raman spectroscopy. The bottom of the chamber is preferred to be composed of Raman compatible material. Suitable examples include quartz glass, CaFl₂ (calcium fluoride) glass or borosilicate glass. It is particularly preferred that the material is translucent. In further preferred embodiment, the chip or parts or it are translucent. Also envisaged are semi-translucent materials. In preferred embodiments, the chip is fabricated from glass by conventional direct laser structuring, powder or sandblasting, or photostructuring. Further eligible materials for the construction of the chip are thermoplastic polymers, such as polymethylmetacrylate (PMMA), polycarbonate (PC), polystyrene (PS), Topas, Zeonor, or Zeonex. The processing technique varies with the material used for the fabrication of the chip. For example, thermoplastic polymers can be process via injection molding, thermoforming, hot embossing, laser machining, or precision mechanical machining. The processing techniques are known in the art and can accordingly be applied by a skilled person. In addition, the chip may be coated, for example, with an SU-8 polymer.

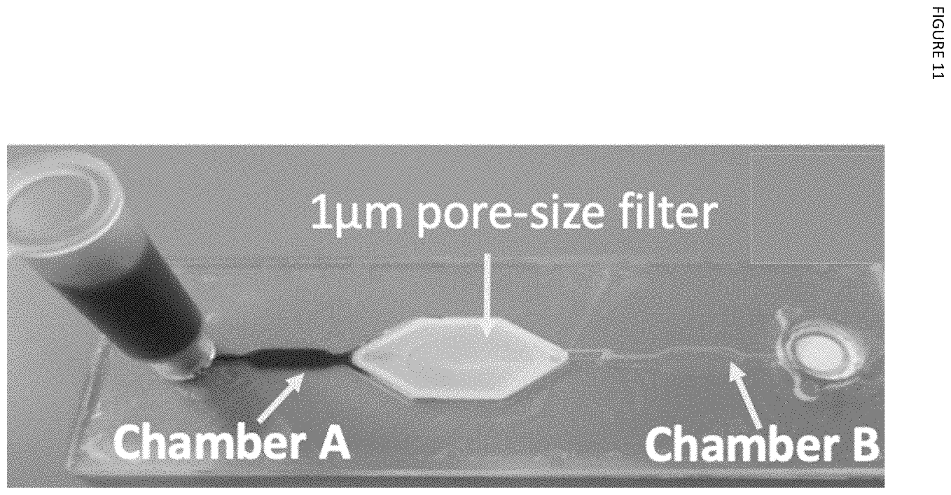

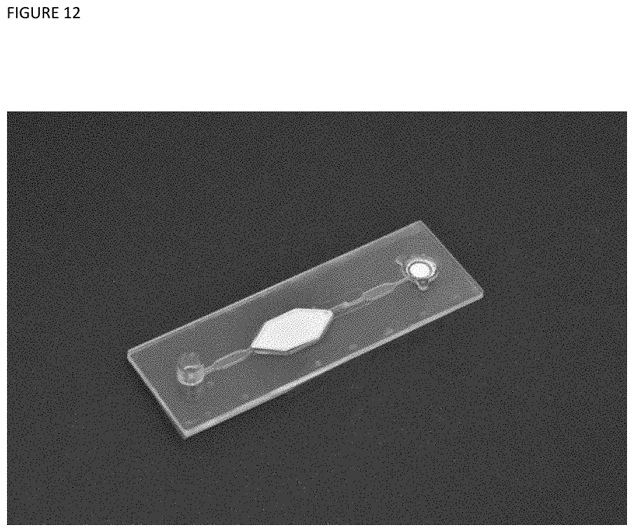

The chip unit may be equipped with a filter membrane as defined herein above. The filtration membrane is typically located downstream from the inlet. The chip may accordingly comprise a filter membrane which is capable of retaining blood cells or other non-microbial cells or particles and thus prevents their entering into inner parts of the chip, in particular into the micro-chambers, while letting pass microbes and smaller particles, e.g. by having pore sizes of about 1.5 to 3 μm (diameter). The filter membrane may be positioned at any suitable central location within the chip to allow for an efficient filtration of samples. Preferably, a filter membrane may be provided in the initial or opening segment of a main channel as defined herein, thus allowing the passage of microbes via the main channel to micro-chambers as defined herein, whereas larger entities such as blood cells etc. are retained in the filter membrane distal to the micro-chambers. Alternatively, the filter membrane may be provided above or in the vicinity of the micro-chambers and thus allow for a direct loading of said chambers through the pores of the membrane. In one embodiment, a filter membrane is provided which comprises a suitable hole or pore above a micro-chamber and hence allows for loading of each of said chambers with microbes separately. It is particularly preferred that the filter membrane is provided as integral part of the chip as defined herein. A non-limiting example of a corresponding implementation can be derived from FIG. 11 and FIG. 12.

In further specific embodiments, the filter membrane allows for a removal of non-microbial particles from the micro-chamber zones of the chip after the filtration process is finished. For example, the filter membrane may be designed as separate layer on top of a chip comprising a multitude of micro-chambers. After the sample has been filtered through said filter membrane and the microbes have entered the micro-chambers, said layer is removed, e.g. by a sliding mechanism. Alternatively, the chip comprising the microbes in the micro-chambers may be moveable and thus be separated from the filter membrane after the sample was filtrated and microbes have entered the micro-chambers.

The chip may, in further embodiments, also comprise a control checkpoint, which typically resides downstream of the filtration unit to check the status and function of the filtration process or filter membranes. In specific embodiments, a micro-channel or micro-chamber located below the filtration unit may be filled with the filtered sample. The termination of this process may, for example, controlled via the presence of semipermeable membranes which are closed once they are in contact with liquids. Also envisaged is an optical and electronic detection mechanism, e.g. via CCD cameras etc., which detects/monitors the filling status of the micro-channel or micro-chamber located below the filtration unit.

Components that are able to pass through the filtration unit and the control checkpoint, may enter the chip, e.g. via one or more of the channels as described herein. Particles or components which are size-excluded by the filter membrane do not enter said channels and are retained on the filter membrane. Further envisaged is a waste or outlet located downstream of the channel, which may be used to evacuate the filtered liquid with the microbe from the channel from the channel for downstream measurements.

In preferred embodiments, the chip is connected to, or integrated into, or part of a microfluidic system. The term “microfluidic system” as used herein relates to a device allowing the precise control and manipulation of fluids that are constrained to small, preferably sub-millimeter scales. Typically, a microfluidic system implements small volumes, e.g. in the range of nl, or pl, and/or it may implement an small overall size. Furthermore, a microfluidic system according to the present invention may consume a low amount of energy. In a microfluidic system according to the present invention effects such as laminar flow, capillary flow, specific surface tensions, electrowetting, fast thermal relaxation, the presence of electrical surface charges and diffusion effects may be implemented and/or used. In certain embodiments, a microfluidic system may have connections with external sources or external elements, e.g. the separation or reservoirs or vessels for reuse purposes may be possible. It is preferred that the system is, at least partially, based on capillary forces. In addition or alternatively, active elements such as micropumps or microvalves may be used. A microfluidic system as envisaged by the present invention may comprise several modules which may be connected by channels. It may further comprise a reservoir for cells and a reservoir for fluids or buffers etc. For the performance of the analysis of a microbial cell, the microfluidic system may comprise a chip with a network of channels, as described herein, which is connected to a Raman spectroscopy system.

The microfluidic system may, in specific embodiments, also comprise zones or modules where nucleic acids can be isolated and analysed, or a module which is configured to allow antibody binding, or an array of microwells allowing for contacting of microbes with a substance, or which allows for cultivation of microbes or any other suitable module or element. Preferably, said channel or zone is configured to slow down liquid movements to allow for optical/spectral analysis of the microbes. In further embodiments, hydrogels, collagen gels or other material which slow down microbes may be used in the system, e.g. within a meshwork of fibers.

Furthermore, the microfluidic system may comprise an electronic or computer interface allowing the control and manipulation of activities in the system, and/or the detection or determination of reaction outcomes. In another specific embodiment of the present invention said microfluidic system may be an integrated microfluidic system. The term “integrated microfluidic system” as used herein refers to the compactation and resizing of the chip in the system, as well as the system itself, e.g. comprising all necessary connections, zones and, optionally, also necessary ingredients within container-like form. The integrated microfluidic system may, for example, have the form of a cartridge and, thus, be entirely closed, or partially closed allowing the introduction of samples, ingredients etc. via resealable inlets. As a cartridge, the system may further be replaceable in an uncomplicated manner. Accordingly, the cartridge may be connected to surrounding units by interfaces which are capable of single step disconnections or simple disruptions. The integrated microfluidic system may further be equipped with alignment structures for optical detection or illumination/stimulation devices. Such a cartridge systems allows for a safe handling of samples which prevents e or contamination of lab technicians or laboratories. Furthermore, the cartridge approach facilitates an easy and comfortable cleaning of the apparatus and/or preparation for further samples to be analysed.

Further envisaged is a unit allowing for the recognition of sample- or ingredient-associated information, e.g. recognition by a scanner of a bar code or matrix codes indicating the sample origin etc., or the identity of provided ingredients, the manufacture date etc. In specific embodiments, the recognition may be implemented via a unit for contactless communication with a base station outside of the system or as part of the control module of the system, which comprises a corresponding reader. Examples of suitable contactless communications units are an RFID (radio frequency identification) unit, preferably a NFC (near field communication) unit, a Bluetooth unit or an ID-chip unit. In typical example, the sample may be tagged with an RFID chip and accordingly be recognized by a suitable RFID reader. Also envisaged is the presence of an interface to a detection unit allowing the electronic or optical determination of analysis outcomes, object/cell positions etc. The chip may further be designed for storage and documentation purposes, e.g. have a geometrical or design element which facilitates storage in a box, refrigerator or safe.

In a further step of the method according to the present invention isolated microbes, preferably present in a suitable zone or micro-chamber of chip as described herein, are analysed spectroscopically by means of spontaneous Raman spectroscopy. The “spectroscopic analysis” as used herein generally relates to the analysis of microbes isolated from a liquid sample by spectroscopic means, i.e. by studying the interaction of one or more microbes and electromagnetic radiation. The determination typically includes interaction with radiative energy as a function of its wavelength or frequency. By stimulating microbial cells, an emission or response of the cells is generated which can subsequently be recorded and analysed. The spectroscopy analysis which is to be performed according to the present invention is “Raman spectroscopy”. This term relates to a spectroscopic analysis which essentially relies on the observation of vibrational, rotational, and other low-frequency modes in a system. The technique is typically used to provide a structural fingerprint of molecules. It relies, in principle, on Raman scattering, i.e. inelastic scattering, of monochromatic light, from a laser in the visible, near infrared, or near ultraviolet range. The laser light typically interacts with molecular vibrations, phonons or other excitations in a system, e.g. a microbial cell, resulting in the energy of the laser photons being shifted up or down. The shift in energy gives information about the vibrational modes in the system. Typically, a sample, i.e. a microbial cell, is illuminated with a laser beam. Electromagnetic radiation from the illuminated entity is collected with a lens and sent through a monochromator. Elastic scattered radiation at the wavelength corresponding to the laser light (i.e. Rayleigh scattering) may be filtered out, e.g. by a notch filter, an edge pass filter, or a band pass filter, while the rest of the collected light is dispersed onto a detector. In a typical embodiment, a Raman spectroscopy system may be used which comprises a light source which can in particular be a laser. The light source is typically configured to output an excitation beam. The excitation beam can for example have a wavelength in the range between 532 nm and 1064 nm, e.g. approximately 785 nm. Subsequently, a Raman spectrometer receives light scattered on the sample, e.g. a cell, by Stokes processes and/or Anti-Stokes processes. Furthermore, the approach may comprise the use of a Raman spectrometer comprising a diffractive element and an image sensor in order to record the Raman spectrum of the sample, e.g. isolated microbe. Furthermore, additional elements may be employed to perform the analysis, e.g. focusing optical elements, which can be designed as lenses, and/or diaphragms. A “spontaneous Raman spectroscopy” means that the objects to be analysed, i.e. microbial cells or the like, are not previously prepared, lysed, processed, dried or otherwise modified in order to allow or facilitate the measurement. Instead, the spontaneous analysis is based on entire cells in their native state, preferably in a liquid, e.g. aqueous environment. This approach allows for an extremely fast and artefact-free analysis, which is not possible if a set of sophisticated preparation steps has to be executed. In addition, measuring single microbes is significantly improved due to optical trapping features, e.g. induced by focusing the Raman excitation laser through the objective of high numerical aperture. In a specific embodiment, an electromagnetic gradient may be induced. Thereby the microbes may be moved towards the central area of the focused laser beam and can be kept there during Raman spectrum acquisition. Further details may be derived from suitable literature sources such as Ashkin, 1970, Phys. Rev. Lett., 24, 156-159; or Ashkin & Dziedzic, 1987, Science, 235, 1517-1520.

The analysis of microbes by means of spontaneous Raman spectroscopy advantageously allows to draw conclusions on the identity and properties of a microbe as defined herein. In preferred embodiments, the determination of spontaneous Raman spectroscopy comprises conducting a statistical evaluation of the at least one Raman spectrum, preferably of a plurality of Raman spectra, e.g. between 10 to 1000 spectra, by means of Raman spectroscopy of the isolated microbes. The plurality of spectra may either be obtained for a single microbe, or for a group of microbes, e.g. one spectrum may be obtained for one microbe. It is particularly preferred to obtain spectra for single microbes, e.g. via the use of optical traps as mentioned herein. It is further preferred that the statistical evaluation is a qualitative determination to which species, genus, family, order or group the microbe or group of microbes belong to.

The statistical evaluation may, for example, be a principal component analysis (PCA) or a cluster analysis for each of the Raman spectra detected. Typically, in the “principal component analysis (PCA)”, a coordinate transformation in the N-dimensional data space is determined in such a way that the analysed entity of data points is spread along its most statistically relevant (e.g. variance-containing) coordinate axes in the transformed coordinate space. These coordinate axes define the principal components. The first principal component PC-1 typically defines the axis with the sharpest differences between the different groups of Raman spectra. “Cluster analysis” relates to a technique to group similar observations into a number of clusters based on the observed values of several variables for each individual. Cluster analysis maximizes the similarity of cases within each cluster while maximizing the dissimilarity between groups that are initially unknown.

Accordingly, by means of a statistical analysis such as the principal component analysis or a cluster analysis, as mentioned herein, it can be determined whether the pattern of Raman peaks contained in the Raman spectrum is characteristic of a specific microbe. Alternatively or additionally, it can be determined whether the pattern of Raman peaks contained in the Raman spectrum is characteristic of the presence of a microbe as a “photonic fingerprint.” A principal component analysis may hence be performed fora Raman spectrum or a plurality of Raman spectra which have been recorded from the sample, e.g. a microbe or group of microbes.

“Linear discriminant analysis (LDA)” or “normal discriminant analysis” or “discriminant function analysis” a dimensionality reduction technique which is commonly used in the pre-processing step for pattern-classification and machine learning applications. The aim of this approach is to project a dataset onto a lower-dimensional space with good class-separability in order to avoid overfitting.

The term “spectral analysis” also refers to the evaluation of characteristic spectral patterns. As such, the determination of whether the Raman spectrum is characteristic of a microbe may hence not be based on individual Raman peaks, but rather on a plurality of Raman intensities distributed evenly or unevenly over the Raman spectra at a plurality of Raman wavenumbers, yielding a characteristic spectral pattern. Thus, by means of a statistical method such as the principal component analysis, as mentioned above, or other statistical methods such as cluster analyses, one can take advantage of the fact that the Raman spectrum as a whole shows characteristics that are indicative and specific of a microbe of a particular species.

The patterns in a Raman spectrum can be defined by one or a plurality of parameters selected from the group composed of the wavenumbers at which the Raman peaks are located, the peak heights, the flank steepness of the peaks, the distances between the peaks, and/or combinations of peaks in one or a plurality of Raman spectra. For evaluation of one or a plurality of Raman spectra detected, e.g. for one microbe, one can determine whether these peak(s) are situated in a space, according to a principal component analysis, in an area assigned to microbes or in another area assigned to non-microbes.

For example, by means of a statistical evaluation, each Raman spectrum can be assigned to a point in an N-dimensional data space, wherein N>>1, e.g. N>100. The N-dimensional data space can be the data space spanned in a principal component analysis by the various principal components. Advantageously, one can determine from reference spectra, e.g. control experiments or previously recorded spectra, in which areas of the N-dimensional data space Raman spectra are arranged in clusters for microbes and in which other areas of the N-dimensional data space Raman spectra are arranged in clusters for non-microbes.

An assignment to the species of microbes can further take place for a cluster analysis or for a different analysis of the recorded Raman spectra for example by means of different wavenumber ranges. For instance, in order to identify Staphylococcus aureus, at least one wavenumber of 1110, 1160 cm⁻¹ and 1525 cm⁻¹ may be detected. In addition at least one wavenumber from one or a plurality of wavenumber ranges of 1650 to 1600 cm⁻¹, from 1350 to 1250 cm⁻¹, from 1180 cm⁻¹ to 1120 cm⁻¹, from 1100 cm⁻¹ to 1050 cm⁻¹, from 930 cm⁻¹ to 890 cm⁻¹ or from 700 cm⁻¹ to 650 cm⁻¹ may be evaluated. In order to perform the cluster analysis, the mentioned wavenumber ranges do not necessarily have to be evaluated, but rather other principal components can also be evaluated.

In a further preferred embodiment, the method comprises a statistical evaluation and judgment on the basis of artificial intelligence and/or machine learning algorithms for complex matrix data evaluation. The term “artificial intelligence” as used herein generally refers to supervised learning approaches. The term includes, inter alia, machine learning concepts. “Machine learning” as used herein typically relies on a twostep approach: first, a training phase; and second, a prediction phase. In the training phase, values of one or more parameters of the machine-learning model (MLM) are set using training techniques and training data. In the prediction phase, the trained MLM operates on measurement data. Example parameters of an MLM include: weights of neurons in a given layer of an artificial neural network (ANN) such as a convolutional neural network (CNN); kernel values of a kernel of a classifier; etc. Building an MLM can include the training phase to determine the values of the parameters. Building an MLM can generally also include determining values of one or more hyperparameters. Typically, the values of one or more hyperparameters of the MLM are set and not altered during the training phase. Hence, the value of the hyperparameter can be altered in outer-loop iterations; while the value of the parameter of the MLM can be altered in inner-loop iterations. Sometimes, there can be multiple training phases, so that multiple values of the one or more hyperparameters can be tested or even optimized. The performance and accuracy of most MLMs are strongly dependent on the values of the hyperparameters. Example hyperparameters include: number of layers in a convolutional neural network; kernel size of a classifier kernel; input neurons of an ANN; output neurons of an ANN; number of neurons per layer; learning rate; etc. Various types and kinds of MLMs can be employed in the context of the present invention. For example, a novelty detector MLM/anomaly detector MLM, or a classifier MLM may be employed, e.g., a binary classifier. For example, a deep-learning (DL) MLM can be employed: here, features detected by the DL MLM may not be predefined, but rather may be set by the values of respective parameters of the model that can be learned during. As a general rule, various techniques can be employed for building the MLM. For example, typically, the type of the training can vary with the type of the MLM. Since the type of the MLMs can vary in different implementations, likewise, the type of the employed training can vary in different implementations. For example, an iterative optimization could be used that uses an optimization function that is defined with respect to one or more error signals. For example, a backpropagation algorithm can be employed. In a further step of the method according to the present invention the susceptibility of isolated microbes, preferably present in a suitable zone or micro-chamber of chip as described herein, to an antibiotic is determined by means of spontaneous Raman spectroscopy, i.e. the Raman spectroscopic technique, including the statistical evaluation, as described above. The determination of susceptibility may preferably be performed at specific zones or areas of a chip as defined herein. For example, these zones or areas may comprise a predefined amount or concentration of a specific antibiotic. The amount or concentration of the antibiotic is typically based on the skilled person's knowledge of the antibiotic's effect on microbes. For example, the concentration may be the MIC (minimal inhibitory concentration), i.e. the lowest concentration of a drug, which prevents visible growth of microbe, or the MIB (minimum bactericidal concentration), i.e. the concentration resulting in microbial death. Accordingly, the duration of antibiotic exposure may, for example, be set in accordance with MIC or MIB parameters. It is preferred that the concentration of the antibiotic is set to a value which is sufficiently high to indicate a reaction of the microbe to it. This value may be higher than the MIC or MIB, e.g. 10%, 25%, 50%, 75%, 100%, 200%, 500% etc. higher. The present invention envisages, in further specific embodiments, additional, different approaches, which make use of different concentrations and/or different exposure times, e.g. multiples of MIC or MIB. These parameters may further be adjusted during the performance of the method.

The measurement may be performed either with the same microbes which have before been analyzed via Raman spectroscopy to determined their identity after the supplementation with antibiotics, e.g. via the microfluidic elements of the invention, or with different microbes.

In preferred alternative embodiments, the analysis of the microbes as to their identity and as to antibiotic susceptibility is performed with two different sub-groups of bacteria. In this embodiment, the microbes which have been isolated from the liquid sample are separated within the chip and/or microfluidic device into two or more separate groups. Assuming that these microbes, deriving from the same sample, belong to the same species or are essentially identical, it is possible to perform, at the same time, or sequentially if required, an analysis with regard to their identity and to their antibiotic susceptibility.

The determination of antibiotic susceptibility of microbes centrally comprises a comparison step of spontaneous Raman spectra obtained for a microbe prior and subsequent to the exposure of the microbe to the antibiotic. “Prior to the exposure to the antibiotic” refers to the acquirement of a Raman spectrum before the microbes come into contact with an antibiotic in specific areas within a micro-chamber the chip. There is no time restraint or limit as to the acquirement of such Raman spectra. The information may, in certain embodiments, have been obtained at any point of time in the past and also be derived from databases or previously recorded spectra or be additionally compared or supplemented with information from previously recorded spectra or database information. “Subsequent to the exposure to the antibiotic” means obtaining a Raman spectrum after the microbes have come into contact with an antibiotic for a specific period of time, e.g. within micro-chamber the chip.

In one embodiment, the microbe may be exposed to the antibiotic for about 0.5 to 30 minutes. Preferably the microbe is exposed to the antibiotic for about 0.5 to 5 minutes, about 5 to 10 minutes, about 10 to 15 minutes, about 15 to 20 minutes, about 20 to 25 minutes, or about 25 to 30 minutes. It is also envisaged to obtain Raman spectra at any other suitable intervals after the exposure to the antibiotic. Preferably the Raman spectra are obtained at intervals of about one minute, about two minutes, about three minutes, about four minutes, about five minutes, about six minutes, about seven minutes, about eight minutes, about nine minutes, or about ten minutes. The intervals may further be combined with changes to the concentration of antibiotics used, e.g. the concentration may be increased or decreased after one or more intervals, e.g. by 5%, 10%, 20%, 50%, 75% or 100%. In further preferred embodiments, the microbes are exposed to one or more gradients of one or more antibiotics. The gradients may be composed of different start and end concentrations and be provided within a micro-chamber as defined herein above, or along a tube or pathway being a part of the microfluidic system, or along a channel being part of the chip as defined herein. It is particularly preferred that the gradients are used with a group of microbes, preferably of the same type or origin, which are located at different positions within the gradient, thus allowing for the determination of the working concentration of an antibiotic. It is preferred to expose the microbes according to the MIC or MIB value for the antibiotic tested. It is also envisaged to obtain more than one Raman spectrum at the different intervals.

In certain embodiment, the microbe is exposed to a single antibiotic, preferably to one of the antibiotics mentioned above. In further embodiments, the microbe is exposed to a combination of at least two different antibiotics. The exposure may be performed simultaneously or sequentially. As used herein, “simultaneously” means a microbe is exposed to a combination of at least two antibiotics at the same time, by preferably using the MIC or MIB of the respective antibiotic, whereas “sequentially” means a microbe is exposed to a first antibiotic followed by exposure to a second or further antibiotic. In further embodiments the antibiotic is provided to the microbe at one or more micro-chambers within the chip. It is envisaged herein that one micro-chamber may contain one antibiotic or a combination of at least two antibiotics.

Upon exposure to the antibiotic, the microbe's cell physiology may be affected at many levels. For example, microbes may respond to the antibiotic by changing their morphology, macromolecular composition, metabolism, and/or gene expression. This typically results in the (gradual) death of the microbe. The changing morphology and physiology thus reflect the microbe's susceptibility to the antibiotic and can be determined by comparing the Raman spectrum prior and subsequently to exposure to an antibiotic. This can typically be detected in a shifting, decrease or increase of peaks in the Raman spectrum, which are specific for a particular microbe in the context to the exposure to an antibiotic. The method of the present invention also envisages a kinetics study illustrating the sensitivity of microbes to an antibiotic or a combination of antibiotics by recording Raman spectra at different intervals. In the case of microbes that are resistant to one or multiple antibiotics, no or slight changes in the Raman spectra are observed upon exposure to the antibiotic or the combination of antibiotics over time.

In a further embodiment of the present invention, the method as described herein additionally comprises a step of quantifying the isolated microbes. As used herein, the term “quantification” relates to the determination of the number of microbes in a liquid sample, e.g. the liquid sample to be analysed according to the present invention. The quantification may typically take place in a confined volume and/or in a defined area, e.g. of the chip as described herein. For example, the quantitation of microbes may be performed in one or more the micro-chambers, or channels etc. of the chip as described herein, i.e. after the sample has been filtered and microbe have been isolated. It is further preferred that the quantification is performed before any cultivation medium is provided or cultivation steps are performed. The quantification may be carried out according to any suitable means. For example, the quantification may be performed by means of cell counting within a part of the chip, e.g. one or more micro-chambers or one or more channels of the chip. The microbes may accordingly be stained or labeled with suitable dyes or fluorescence labels known to the skilled person. The image analysis may be performed with a microscope. In corresponding embodiments, the microbes are preferably arrested in suitable materials which slow down their movement such as hydrogels or collagen gels. The present invention thus also envisages a method wherein in the analysis of step b) and/or the determination of step c) as described herein comprises collecting a microbe, slowing down movement of said microbe and optionally arresting said microbe in a decelerating material for different purposes, in particular for recording a Raman spectrum. Suitable examples of such material are a fibrous gel, a hydrogel or collagen gel.

A microbe which has been visualized and/or morphologically been determined may be marked under the microscopic view as counted or cell of interest. Such a marking may, preferably, be a virtual marking or be based on the use of a virtual label. Typically, such a marking may be implemented by a computer-based or software solution, which records a picture of microbes and highlights a microbe of interest. Such a microbe may subsequently be tracked, e.g. if the microbe is moving or floating in a group of microbes. The determined quantity of microbes per sample volume, optionally in combination with the information on the identity of the microbes, allows for a diagnostic statement as to the health state of a patient. For example, is the determined quantity within the sample increased, e.g. by up to 50%, e.g. by 10%, 20%, 30%, 40%, in comparison to a control sample, e.g. derived from healthy patient or a reference value derivable from a database, a microbial infection may be inferred. In such a case, also the possibility of sepsis may be given and the sample and patient may be further examined, e.g. for corresponding symptoms. Further, is the determined quantity within the sample strongly increased, e.g. by 50%, 100%, 150%, 300%, 500%, 1000% or more, or any value in between the mentioned values, in comparison to a control sample, e.g. derived from a healthy patient, or a reference value derivable from a database a sepsis or a high likelihood for a sepsis may be inferred. Accordingly, the present invention also envisages an in vitro method for the determination of microbial infection, microbially caused sepsis or an increased likelihood for microbially caused sepsis. Such a method may, for example, essentially comprise at least steps (a) and (b) of the method for analyzing a liquid sample as described herein. In further embodiments, the present invention also envisages an in vitro method as defined herein for the determination of antibiotic treatment options for microbial infections or microbially caused sepsis. Such a method may, for example, essentially comprise at least steps (a) and (b) and (c) of the method for analyzing a liquid sample as described herein.

To facilitate the analysis of microbes and the determination of antibiotic susceptibility of the microbes as described above, a microbe may be transported or moved within the chip or microfluidic system, be collected, and/or arrested, e.g. in a micro-chamber, with the help of an optical trap. This further allows to suitably record a Raman spectrum of the trapped microbes. The term “optical trap” as used herein relates to a single-beam gradient force trap or optical tweezer, which uses a highly focused laser beam to provide an attractive or repulsive force. The optical trap may be produced by the excitation beam of the Raman spectroscopy system or a beam of electromagnetic radiation different therefrom. For example, a focal point of a beam may produce an optical trap potential, in which a cell is collected for the Raman spectroscopy. The focal point can be produced by the excitation beam, which is output by a light source. In such an embodiment, the excitation beam can thus be used both as excitation for the Raman scattering and for producing the optical trap. Alternatively, the optical trap can also be produced by a separate beam. The term “arrest” as used herein relates to a brief holding of a cell at a specific position to allow for the performance of Raman spectroscopy. In order to move the microbes within the channel or chamber of the chip or to transport them towards a microfluidic stream as mentioned herein it is particularly preferred to use a pulse of the Raman excitation laser. In specific embodiments, this pulse is used for catapulting or rapidly moving the microbes. Alternatively, a pulse from a UV laser (e.g. a 332 nm N-Laser) may be used.

In a preferred embodiment of the method as described above, the method comprises a comparison of the Raman spectrum obtained from the isolated microbe with a reference spectrum, thereby determining the identity of said microbe. The term “reference spectrum”, as used herein, relates to a Raman spectrum obtained from a microbe of known identity to be used as a matching template in order to designate a relation to a Raman spectrum obtained from a microbe of unknown identity, thereby identifying the unknown microbe. The spectrum may, for example, have been obtained previously or simultaneously from a control experiment. The control experiment may, for example, be performed with a predefined number of microbes whose identity and/or properties are known, e.g. derived from strain collection sites such as ATCC or DSMZ, or which have previously been determined and are cultivated for control purposes. For example, control microbe, in particular bacteria, may be derived from the following groups: Achromobacter, Acinetobacter, Brucella, Cyanobacterium, Pseudomonas, Helicobacter, Escherichia, Salmonella, Shigella, Enterobacter, Klebsiella, Listeria, Serratia, Proteus, Oligoflexia, Campylobacter, Haemophilus, Morganella, Vibrio, Shigella, Spirochaeta, Treponema, Wolbachia, Yersinia, Stenotrophomonas, Brevundimonas, Ralstonia, Fusobacterium, Prevotella, Branhamella, Neisseria, Burkholderia, Citrobacter, Hafnia, Edwardsiella, Aeromonas, Moraxella, Pasteurella, Providencia, Staphylococcus, Streptococcus, Legionella. Particularly preferred are the following species or subspecies Acinetobacter baumannii, Bacteroides fragilis, Bordetella japonica, Devosia pacifica, Enterobacter cloacae, Flavobacterium akiainvivens, Gluconacetobacter diazotrophicus, Haemophilus haemolyticus, Hemophilus influenza, Klebsiella pneumoniae, Legionella pneumophila, Moraxella bovis, Neisseria gonorrhoeae, Proteus mirabilis, Pseudomonas aeruginosa, Rickettsia rickettsii, Salmonella enterica, Serratia marcescens, Vibrio cholera, Staphylococcus aureus, Staphylococcus epidermidis, Streptococcus pneumoniae, Haemophilus influenzae, Escherichia coli, EHEC, Salmonella spp. and Neisseria meningitidis. In further embodiments, the control microbe may be derived from eukaryotic unicellular fungi such as Kluyveromyces, Pichia, Saccharomyces or Candida, e.g. Candida albicans. Alternatively, the control microbe may be a protist such as a flagellata, ciliphora or sporozoa, e.g. Plasmodium, Trypanosoma, Entamoeba, Balantidium, Amoeba, Syringammina, Bodo, or Nocto.

In certain embodiments, one or more of the above mentioned microbes or any other suitable microbe may be provided in the chip, e.g. in one or more of the micro-chambers and be analysed together with the microbes isolated from the liquid sample. Subsequently, a comparison of the obtained Raman spectra may be performed. The microbes may be provided in the micro-chambers in a fixed form, e.g. via a PFA fixation. Further details would be known to the skilled person or can be derived from suitable literature sources such as Tabah et al., 2012, Intensive Care Med, 38, 1930-1945.

In case the method is used for the detection of sepsis, it is preferred to use for the control experiments microbes which are typically involved in the development of sepsis such as Streptococcus pneumoniae, Haemophilus influenzae, Staphylococcus aureus, in particular MRSA, Escherichia coli, Salmonella spp. and Neisseria meningitidis. In preferred embodiments, the control experiments are performed before the samples are analysed. Correspondingly obtained values are preferably stored in a Raman spectrum database and can be compared with sample data obtained in accordance with the herein described methodology. In specific embodiments, third party control samples may be used, e.g. Bioballs as marketed by Biomerieux. These samples may advantageously be entered into micro-channels or micro-chambers of the chips of the present invention. This allows for a skipping of any filtration step as described herein.

In a further preferred embodiment, the reference spectrum is derived from a database, e.g. an organized collection of Raman spectra obtained from a multitude of different microbe species, e.g. those mentioned above, stored and accessed electronically from a computer system. The database may further comprise spectral information on previously measured spectra of control microbes which were exposed to one or more antibiotics as mentioned herein. For instance, microbial samples could be measured by Raman spectroscopy to generate a Raman data library of defined native microbes (i.e. samples in their natural environment, i.e. in solution). In a second step, unknown species could be measured and the resulting data compared with the data library to specify the species of the bacteria present in the sample.

In a further particularly preferred embodiment, the determination of microbial infection is performed in an automated or semi-automated manner. To be capable to determine microbial infection automatically or semi-automatically, method steps as mentioned herein above may be performed in a computer-based manner. For instance, once microbes enter a detection, e.g. of a microfluidic system as described above, images may be acquired. By using suitable image analysis software and/or cell tracking or cell counting devices and/or software, specific microbes may be recognized, highlighted and/or be virtually labelled. The corresponding activities may be performed automatically, or, in certain embodiments semi-automatically, e.g. by requiring a human interaction or by asking for confirmation by the operator. Upon completion of these steps, additional analysis steps may automatically be started such as performance of stimulation of the microbes, spectral, e.g. Raman analyses, recording of spectra, e.g. Raman spectra, recording of bright field images of microbes, fluorescence of microbes, classification of microbes, quality control checks, comparison steps with visual images etc. Correspondingly obtained information may further be accumulated, stored in suitable databases or on suitable servers, transferred to remote systems or entities etc. It is preferred that all images taken are saved on a local hard disk and/or on a cloud server, at least until a sample or group of microbes has entirely been analysed. The saving time may further be extended for documentation purposes.

In further embodiments, the automatic determination may comprise a scanning activity, wherein preferably a predefined number of Raman spectra are collected automatically in a defined area. It is thus preferred that the concentration of bacteria is set or kept at a suitable, typically high value so that with switching on the laser one microbe is caught, the Raman spectrum is taken. Subsequently, the laser may be switched-off and the system may move to a different position, e.g. in a predefined distance, where the steps are repeated, i.e. the laser is switched on, a new sample is arrested, then measured and released etc. The defined area may, for example, but a subportion of the zone where the microbes are located. By scanning a defined area, it is possible to determine how many microbes are present within the area. The scanning approach may be connected with the addition of a virtual label to each microbe, i.e. a tracking activity. The scanning may include the performance of spectral analyses as defined herein, e.g. Raman spectroscopy as mentioned above.

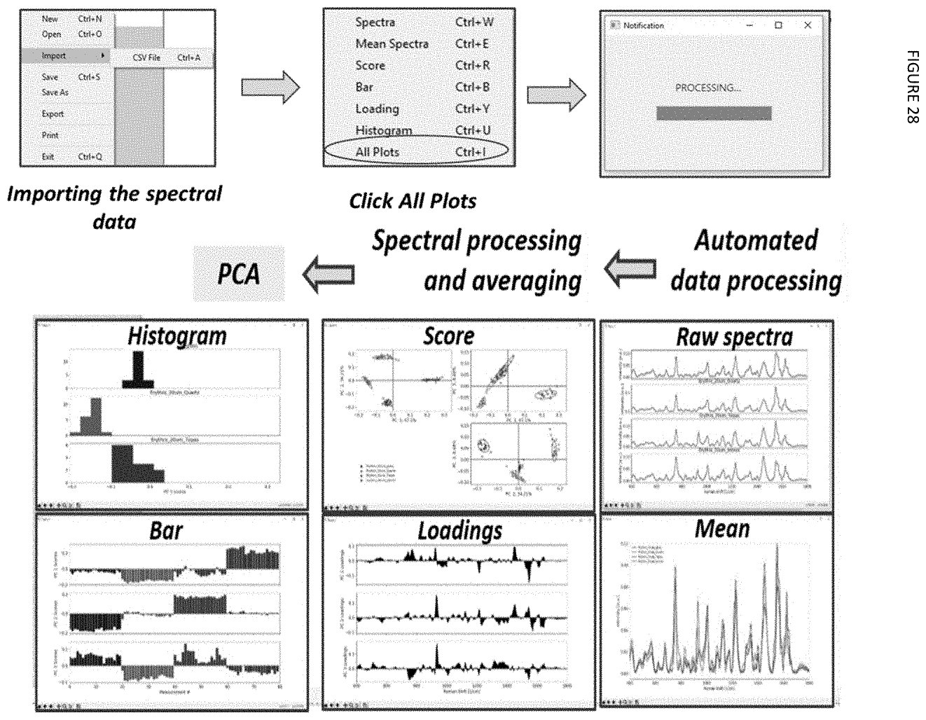

In a particularly preferred embodiment, the analysis is performed by suitable and unique data analysis software, e.g. CT-RamSES, which is capable of processing and analysing Raman spectra taken from biological samples. It is preferred that the data analysis software provides fast spectral processing, safe data storage and easy statistical data analysis for biomedical data interpretation. For example, spectral data are imported from a control software and are subsequently automatically processed by the data analysis software. The software accordingly provides the data analysis plots. The underlying process includes organizing raw spectra of different data sets after conducting all spectral processing steps of (i) Smoothing (noise and cosmic spike removal) (ii) baseline corrections (intrinsic glass-back ground scattering removal) (iii) vector normalization (laser and instrumental effects removal, standardizing all spectra). Subsequently, mean Raman spectra with standard deviations can be calculated for each data set separately. Subsequently, principal components analysis may be conducted on the processed data sets, resulting in score plots describing the similarity and differences between the analysed data sets in form of a scatter plot. In a further embodiment, loadings of principal components may be presented in many plot forms: loadings peaks, bar, and histogram, indicating the spectral variations between the data sets that have been used in the analysis. These spectral variations are assigned to its respective biochemical changes. In a further embodiment, cluster analysis using K-means is designed and used to classify all measured spectra into groups of similar patterns, which can be used to identify diversities and subclasses within one measured heterogeneous sample. Further information may be derived from FIG. 28.

In a specific set of embodiments the present invention relates to an in vitro method for discriminating microbes spectroscopically comprising: a) isolating microbes from a liquid sample as defined herein above; b) analysing said microbes spectroscopically by means of spontaneous Raman spectroscopy as defined herein above. Microbes which may be discriminated are those mentioned herein above. The discrimination may be a discrimination between species, subspecies or strains.

In a further specific set of embodiments the present invention relates to an in vitro method for detecting pathogenic microbes spectroscopically comprising: a) isolating microbes from a liquid sample as defined herein above; b) analysing said microbes spectroscopically by means of spontaneous Raman spectroscopy as defined herein above. Microbes which may be discriminated from other microbes or which may be detected are preferably the following species or subspecies Acinetobacter baumannii, Bacteroides fragilis, Bordetella japonica, Devosia pacifica, Enterobacter cloacae, Flavobacterium akiainvivens, Gluconacetobacter diazotrophicus, Haemophilus haemolyticus, Hemophilus influenza, Klebsiella pneumoniae, Legionella pneumophila, Moraxella bovis, Neisseria gonorrhoeae, Proteus mirabilis, Pseudomonas aeruginosa, Rickettsia rickettsii, Salmonella enterica, Serratia marcescens, Vibrio cholera, Staphylococcus aureus, Staphylococcus epidermidis, Streptococcus pneumoniae, Haemophilus influenzae, Escherichia coli, EHEC, Salmonella spp. and Neisseria meningitidis, or sub-species thereof, or variants or strains thereof.

In a further aspect, the present invention relates to a device for analysing a liquid sample as to the presence, identity and properties of microbes, wherein the device comprises as a first unit (i) a chip comprising a filtering unit and an antibiotics exposure unit capable of determining the susceptibility of microbes to an antibiotic; as a second unit (ii) a Raman spectroscopy system; and as a third unit (iii) an evaluation module which is coupled to the Raman spectroscopy system. Also envisaged is a device for analysing a microbe or a cell being or having been in direct or indirect contact with a microbe in a liquid sample comprising as a first unit (i) a visualization system; as a second unit (ii) a Raman spectroscopy system with combined integrated simultaneous trapping features; and as a third unit (iii) an evaluation module which is combined to the Raman spectroscopy system.

It is preferred that the device comprises a chip as defined herein above in the context of the methods of the present invention.

The second unit of the device, i.e. the Raman spectroscopy system, may comprise a light source which can in particular be a laser. The light source is configured to output an excitation beam. The excitation beam can for example have a wavelength in the range between 532 nm and 1064 nm, e.g. approximately 785 nm. A Raman spectrometer receives light scattered on the sample, e.g. a cell as defined above, by Stokes processes and/or Anti-Stokes processes. The Raman spectrometer can comprise a diffractive element and an image sensor in order to record the Raman spectrum of the sample. The Raman spectroscopy system can comprise further elements in a manner known per se, for example focusing optical elements which can be designed as lenses, and/or diaphragms.

In a further preferred embodiment, the device according to the present invention comprises an integrated optical trapping module. The optical trapping module is able to produce an optical trap for collecting and arresting a microbe therein, in order to record a Raman spectrum. The optical trap can be produced by the excitation beam of the Raman spectroscopy system or a beam of electromagnetic radiation different therefrom. The excitation beam can thus be used both as excitation for the Raman scattering and for producing the optical trap. Alternatively, the optical trap can also be produced by a separate beam. The Raman spectroscopy system can also comprise a light conductor, for example an optical fibre, by means of which the excitation beam and/or the Raman scattered light is guided. The light conductor can be positioned such that the excitation beam leaving said light conductor produces the optical trap with a focal point. In further specific embodiments, the optical trap may be split into several beams to simultaneously trap a multiple number of microbes.

The third unit of the device, i.e. the evaluation module, can be a computer or can comprise a computer. The evaluation module may be coupled to the Raman spectroscopy system and/or the microscope system as defined herein above. The evaluation module can control the recording of the Raman spectrum by the Raman spectroscopy system, as well as the visual and/or fluorescent recording of the microbes. In addition, the evaluation module comprises an interface in order to receive data from an image sensor of the Raman spectroscopy system or the microscope system. The evaluation module may comprise an integrated semi-conductor circuit which can comprise a processor or controller and which is configured to evaluate the recorded images or Raman spectra in order to determine the identity of a microbe or group of microbes. The integrated semi-conductor circuit is configured to determine by means of the Raman spectrum, optionally in combination with interpretation of visual images, the presence, identity and properties of a microbe. The integrated semi-conductor circuit as mentioned above can be configured to identify the presence or absence of determined Raman peaks or to determine the spectral weight of Raman peaks which relate to the identity of a microbe.

In a further preferred embodiment, the evaluation module is designed to perform a statistical evaluation and judgment on the basis of artificial intelligence and/or machine learning algorithms for complex matrix data evaluation. A corresponding evaluation makes use of methods for artificial intelligence and/or machine learning algorithms for complex matrix data evaluation as described herein above. It is preferred that training data are obtained from previous, e.g. supervised, analyses and/or are derivable from databases as described herein.

The evaluation module can also comprise an optical and/or acoustic output unit, via which the information dependent on the analysis of the Raman spectrum is output, which shows, for example, whether or not antibiotic susceptibility of a microbe has been identified. The output unit can also be structurally integrated into a housing of the evaluation module or of the Raman spectroscopy system.

The evaluation module can further comprise a memory in which comparative data is stored which the integrated semi-conductor circuit can use when evaluating the Raman spectrum. Information regarding the position and/or the spectral weight of different Raman peaks for analysed cells can be stored in a non-volatile manner in the memory of the module. Alternatively or additionally, the information regarding the position and/or the spectral weight of different Raman peaks for the analysed microbes can be determined by the module by means of methods of supervised learning or other machine learning techniques.

In a further embodiment, the device according to the present invention additionally comprises as a fourth unit a microfluidic component for semi-automated measurement and/or transporting and/or separating a liquid sample or microbes, which is coupled to the Raman spectroscopy system. The microfluidic component may essentially comprise the elements and components as described above in the context of the microfluidic system mentioned in the methods of the present invention. The microfluidic component may, for example, be configured to allow semi-automated or automated measurement of microbes. It may in addition or alternatively be configured to transport a liquid sample, culture medium, waste, size-excluded particles or microbes. It may further or alternatively be coupled to the evaluation module as defined herein above and/or the control checkpoint, which typically resides downstream of the filtration unit, in the chip as described above. Briefly, it may allow for a precise control and manipulation of fluids. It may further comprise active elements such as micro-pumps or microvalves. It may further comprise a reservoir for microbial cells and a reservoir for fluids or buffers etc. It may additionally enable the isolation and collection of a microbe of interest, e.g. for further analysis, or cultivation or breeding, e.g. for further examination in the future or with an increased number of cells. Envisaged analysis options include, for example, PCR analysis, analysis on DNA microarrays, or sequencing analysis, e.g. via next generation sequencing or nanopore sequencing.

A further aspect of the invention relates to a system comprising the device and a module comprising a database comprising reference values of Raman spectra obtained from microbes. Said module refers to an integrated database of reference values of Raman spectra that were obtained from a previous or simultaneous control experiments.

The control experiments may comprise, for example, identifying microbes with conventional methods known in the art, such as culturing or MALDI-TOF, and subjecting the identified microbes to Raman spectroscopy to record the respective Raman spectra. Said Raman spectra may then be fed into a database and used as comparative reference spectra for identifying microbes from liquid samples.

In a further aspect the present invention relates to the use of the method as defined herein above, of the device as defined herein above or of the system as defined herein above for the detection of sepsis in a subject. In a particularly preferred embodiment the present invention relates to the use of the method as defined herein above, of the device as defined herein above or of the system as defined herein above for the detection of sepsis and antibiotic susceptibility of the sepsis' causative agents.

In a further specific set of embodiments the present invention relates to the use of the device as defined herein above or of the system as defined herein above for detecting pathogenic microbes spectroscopically by means of spontaneous Raman spectroscopy as defined herein above.

In a further specific set of embodiments the present invention relates to the use of the device as defined herein above or of the system as defined herein above for discriminating microbes spectroscopically by means of spontaneous Raman spectroscopy as defined herein above.

Microbes which may be detected or discriminated from other microbes are preferably the following species or subspecies Acinetobacter baumannii, Bacteroides fragilis, Bordetella japonica, Devosia pacifica, Enterobacter cloacae, Flavobacterium akiainvivens, Gluconacetobacter diazotrophicus, Haemophilus haemolyticus, Hemophilus influenza, Klebsiella pneumoniae, Legionella pneumophila, Moraxella bovis, Neisseria gonorrhoeae, Proteus mirabilis, Pseudomonas aeruginosa, Rickettsia rickettsii, Salmonella enterica, Serratia marcescens, Vibrio cholera, Staphylococcus aureus, Staphylococcus epidermidis, Streptococcus pneumoniae, Haemophilus influenzae, Escherichia coli, EHEC, Salmonella spp. and Neisseria meningitidis. Microbes which may be discriminated are those mentioned herein above. The discrimination may be a discrimination between species, subspecies or strains.

The figures and drawings provided herein are intended for illustrative purposes. It is thus understood that the figures and drawings are not to be construed as limiting. The skilled person in the art will clearly be able to envisage further modifications of the principles laid out herein.

### EXAMPLES

### Example 1

### Fast Raman Measurements of Individual Bacteria Using Optical Trapping Features

Raman trapping microscopy allows for fast detection and characterization of bacteria. Due to the implemented Trapping features specimen are captured at the laser focus and hold tight during Raman analysis. The focused laser beam induces high photon density—creating a strong electromagnetic field gradient for optimal trapping, as well as resulting in spectra of high intensity. This combination results in good and reliable spectra even of motile samples and has opened a new venue of applications especially for samples in solution in the sub-micrometer scale such as bacteria or exosomes.

Raman detection enhancement: Many approaches were developed to enhance Raman signals such as using Plasmon/resonance effects in Surface-Enhanced Raman Scattering (SERS), to enable Raman measurements of small cells. However, it requires chemical modification of the sample (applying nanostructures) and special coatings of the substrate surface. Thus, it is a sample destructive and time intense analysis.

In contrast, due to raise of spectral intensity (>>10 fold) in trapped samples, BioRam® analysis bacteria and cells in minutes—direct within their native environment and in a highly automated manner.

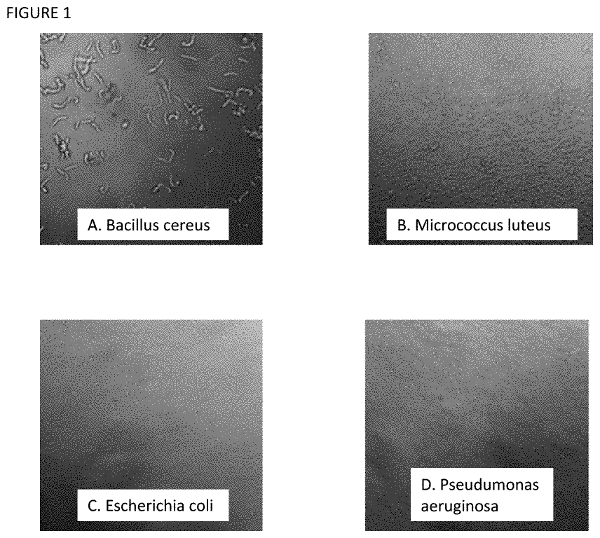

The Raman trapping of a small number of single bacteria was shown to provide enough spectral information to differentiate bacterial species. Four species—Bacillus cereus, Micrococcus luteus, Pseudomonas aeruginosa, Escherichia coli (see FIG. 1 for microscopy pictures of the morphology of the four bacteria samples)—were pipetted into the channel of a channel slide. Only 10 cells of each species were measured.

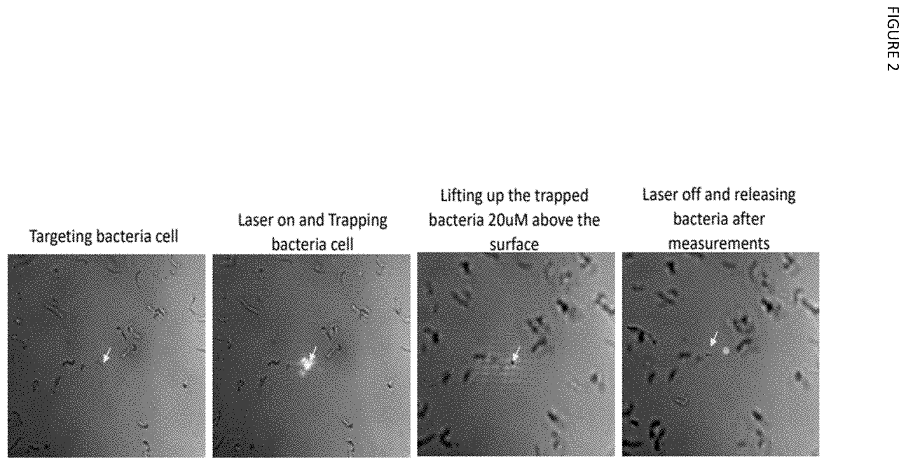

FIG. 2 provides details on the trapping procedure (from left to right), wherein (i) a bacterial cell is targeted; (ii) the Raman excitation laser is switched on (bright spot), simultaneously a bacterium is trapped; (iii) by changing the microscope focus, the trapped bacterium is lifted in the z direction; (iv) moving the microscope stage in x/y direction causes the bacterium to move accordingly; (v) finally, switching off the laser releases the bacterium which moves away unaffectedly

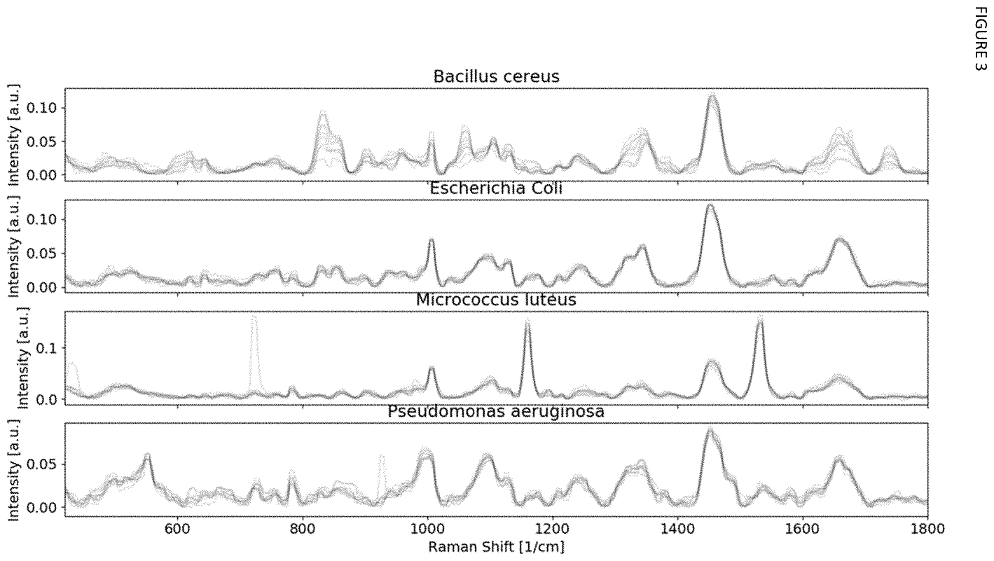

Due to the high photon density the bacteria could be clearly discriminated from each other. The Raman mean spectra were calculated, displaying characteristic spectral patterns of each species. FIG. 3 shows overlay plots of correspondingly processed Raman spectra of the measured four bacteria samples. The bacteria correspond to those shown in FIG. 1. Each single thin line represents one Raman spectrum of one single bacterium.

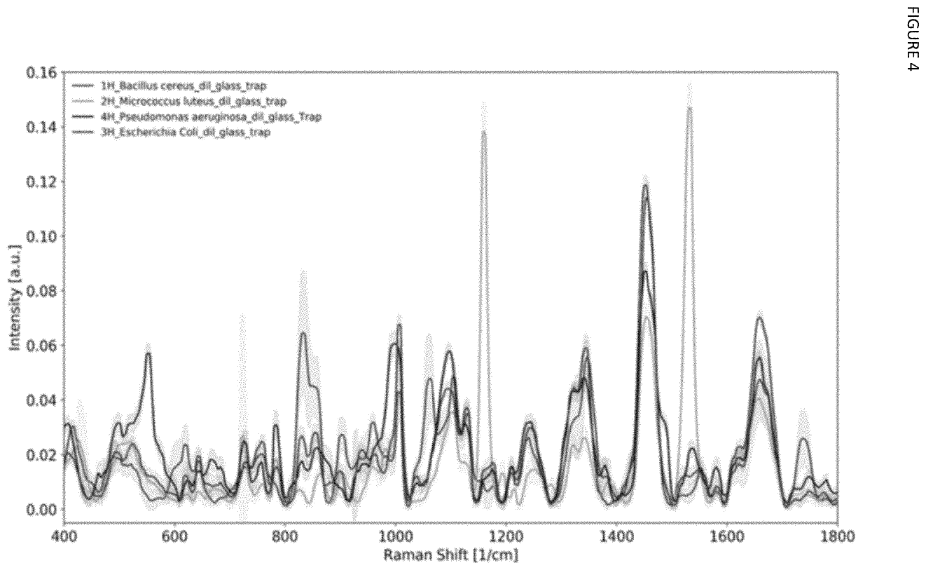

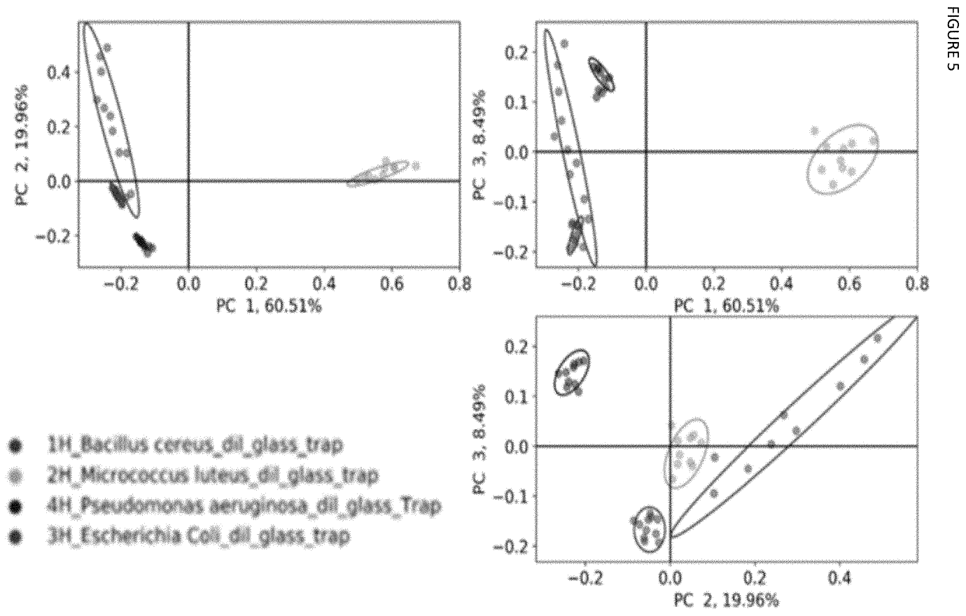

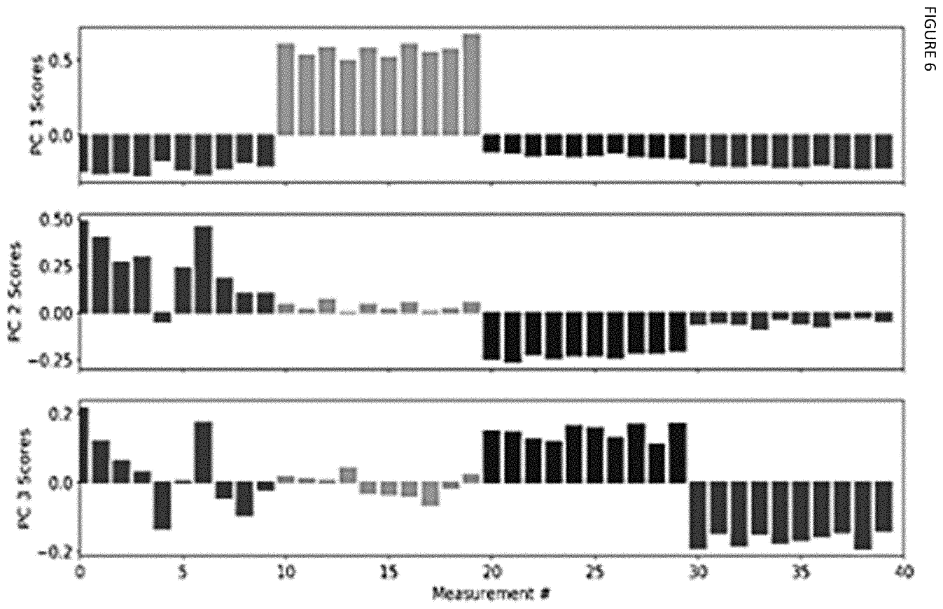

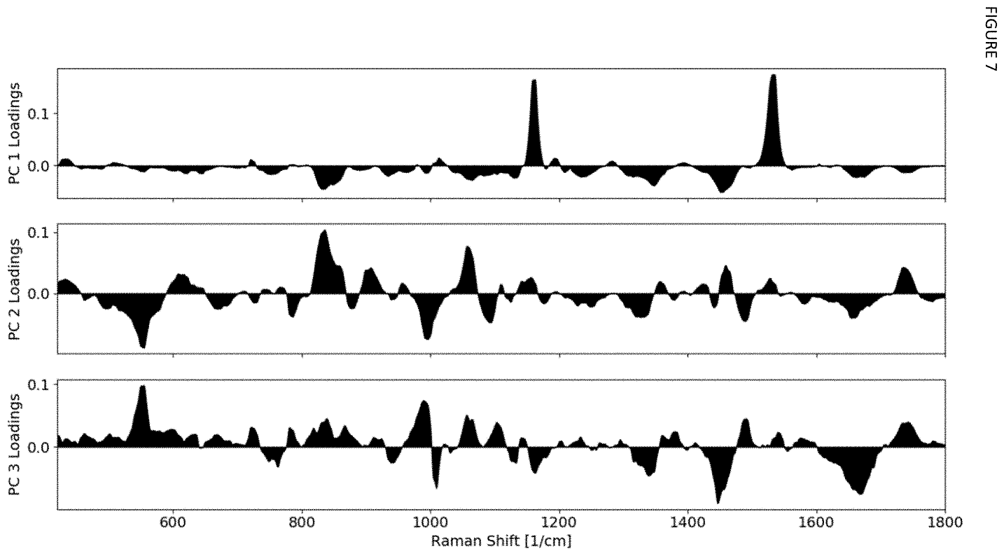

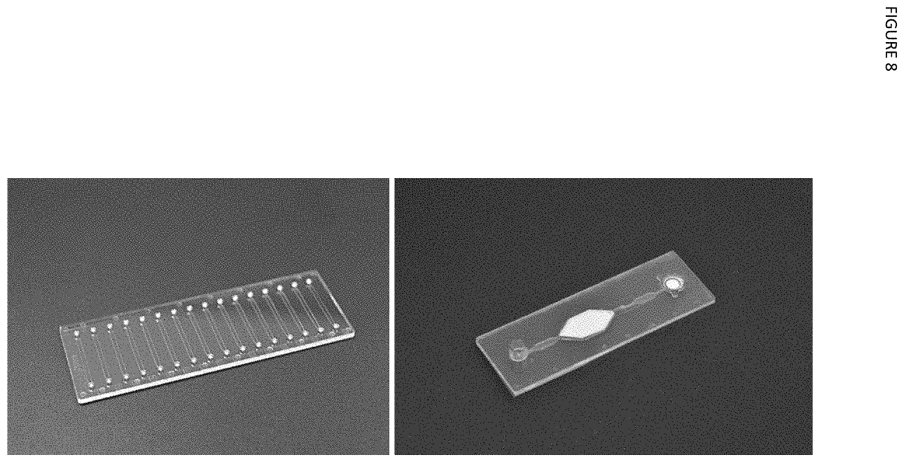

Further processing and analysis steps can be derived from FIG. 4, which depicts an overlay of the mean spectra of the four bacteria samples depicted in FIG. 3 and FIG. 5, which shows principal component analysis (PCA) score plots of all the measured data from the four bacteria samples depicted in FIGS. 3 and 4. Finally, FIG. 6 shows bar plots of the second and third two principal component of all measured data, while FIG. 7 depicts loading plots of the first two principal component of all measured data.

In summary, the fast Raman measurements of individual bacteria using optical trapping features as described herein was fully capable of distinguishing the tested species Bacillus cereus, Micrococcus luteus, Pseudomonas aeruginosa and Escherichia coli.

### Example 2

### Detection of Microbes in Lettuce Extract Using Simultaneous Raman Trapping Microscopy

The Raman spectroscope-microscope system could be a suitable tool for fast identification of bacterial contamination in food—such as iceberg salad.

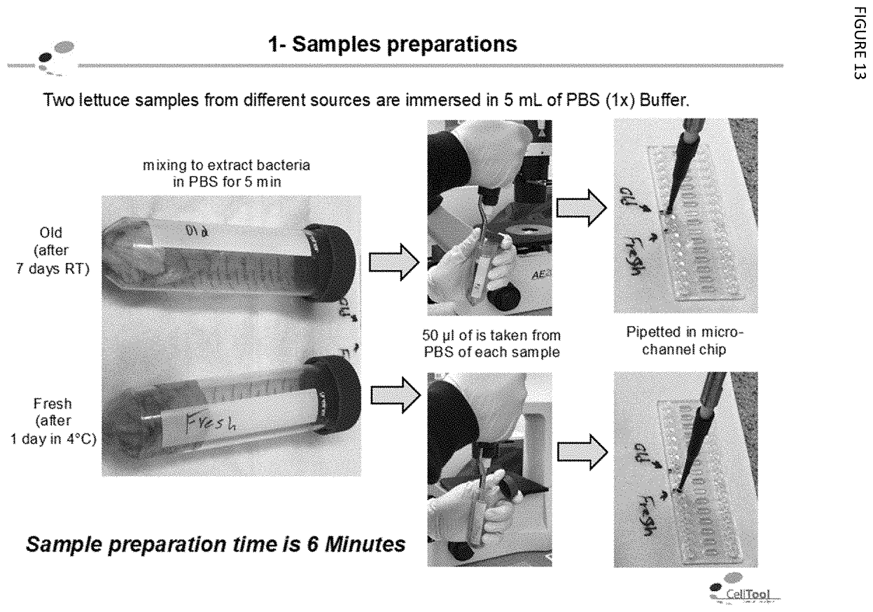

Two lettuce samples from different sources (a few leaves of fresh and 7 days old Iceberg salad) were immersed in 5 ml of PBS (1×) buffer and mixed for 5 min (see also FIG. 13 left). Subsequently, 50 μl were taken from each sample mixture and pipetted into microchannel chips (see also FIG. 13 right). Raman measurements of single bacteria were performed.

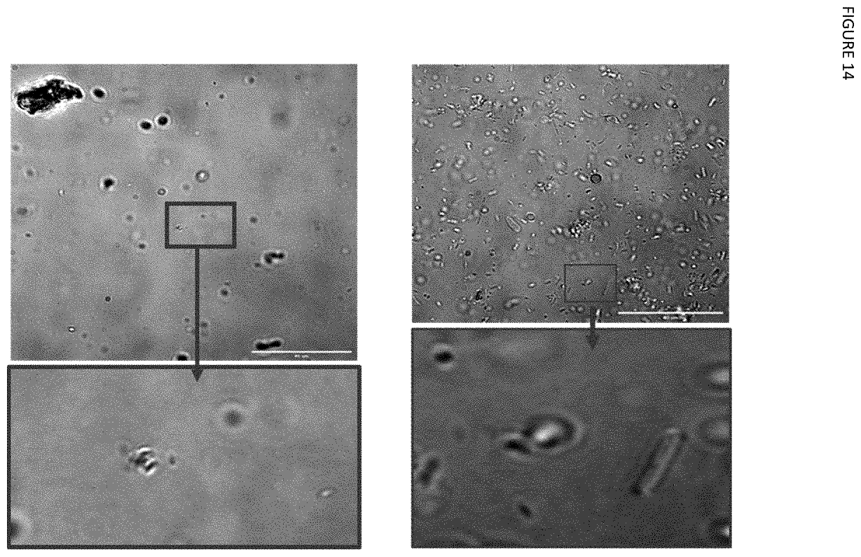

As can be derived from a microscopic view of microbes from fresh and 7 days old lettuce (see FIG. 14 left and right), the amount of microbes is much higher in old lettuce. In addition, different morphologies varying from small round bacteria up to rod like fungi were observed.

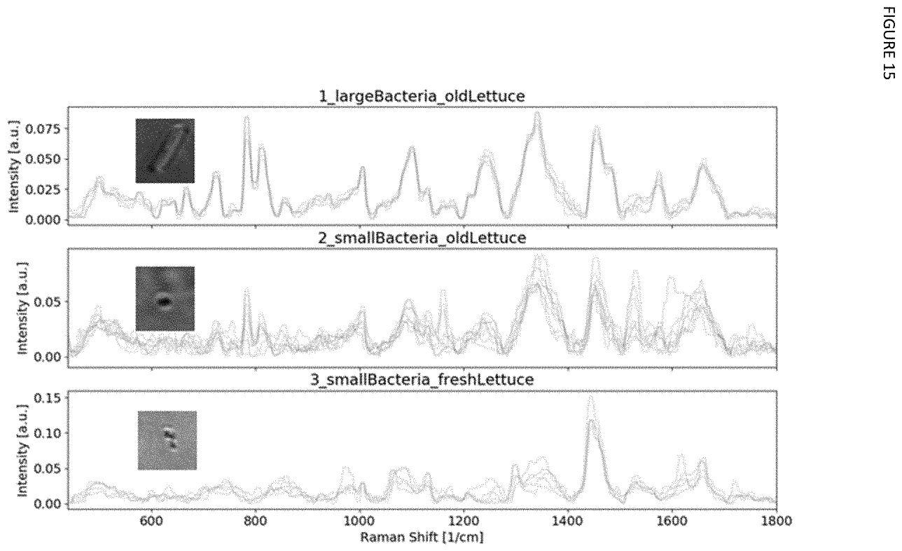

The correspondingly performed Raman spectra further revealed clear differences in the spectral patterns between the fresh and 7 days old lettuce (see FIG. 15 left and right).

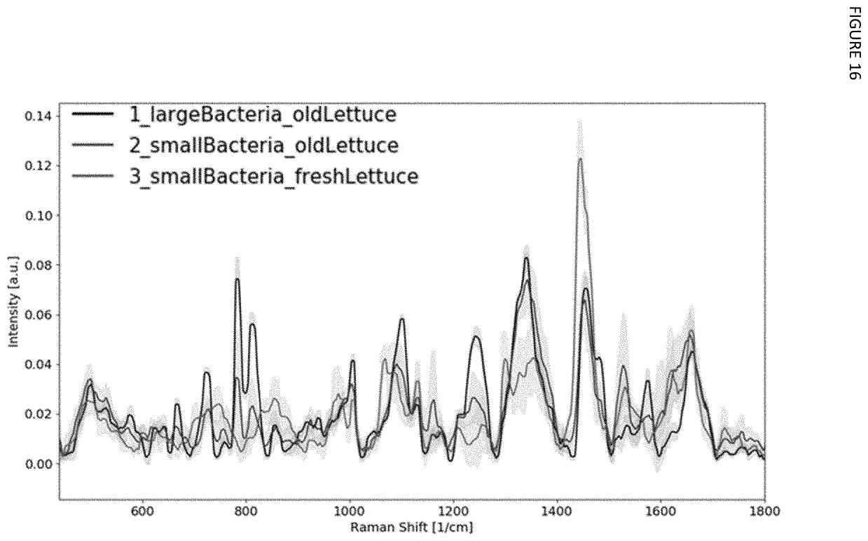

An overlay of the mean Raman spectra as depicted in FIG. 16 show the differences between the microbes even more clearly.

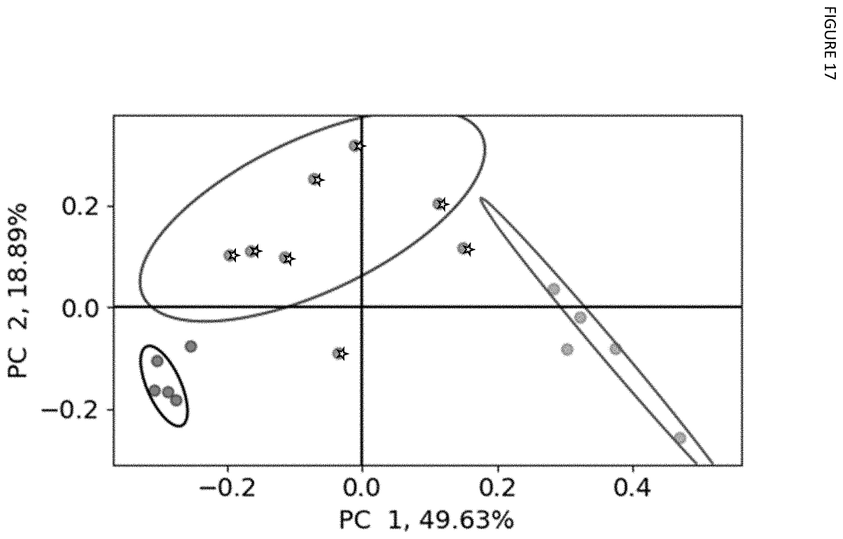

As can be observed in FIG. 17 after Principal component analysis (PCA) the score plot demonstrates the differences (in this preliminary test only 5 cells per samples were measured).

### Example 3

### Detecting the Effect of Antibiotic on Bacteria Using Raman Trapping Microscopy

Live bacteria have been collected from Sputum and separated to two groups:

1—Untreated bacteria (control); and
2—Bacteria treated with ofloxacin (2 mg/mL) for 2 hrs.

The aim of this experiment is to show if the Raman trapping microscopy, in particular the BioRam apparatus, can detect the effect of the antibiotic on the bacteria spectra, which can be used as indicator if the bacteria is responding to the antibiotic treatment.

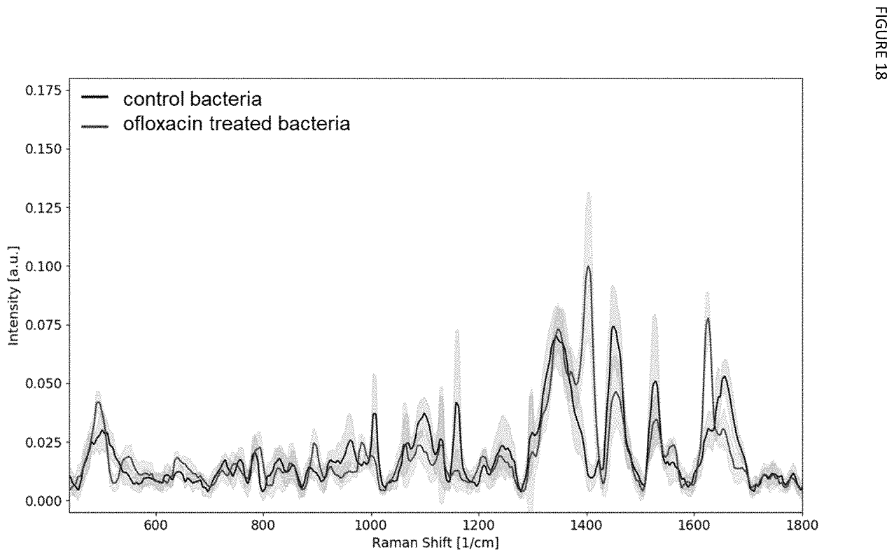

As can be derived from FIG. 18, Raman spectra of bacteria treated with ofloxacin (shown as star-like dots) the same bacteria without any treatment (control—shown in black), Raman spectra changes are clearly observed.

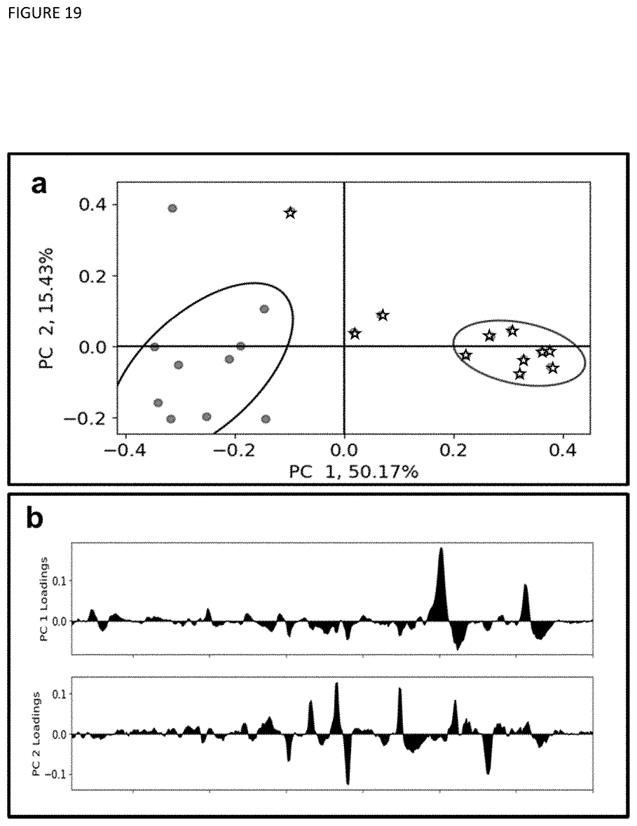

According to a principal component analysis (PCA) of the Raman data as derivable from FIG. 19, a clear difference between control and ofloxacin treated bacteria can be observed. In FIG. 19 (a) the score plot revealing different scattering patterns between control bacteria (black dots) and bacteria treated with ofloxacin (star-like dots). In FIG. 19 (b) the loading plots illustrate the difference of the Raman bands that were used for classifications.

### Example 4

### Fast Identification of Contaminated Blood Products

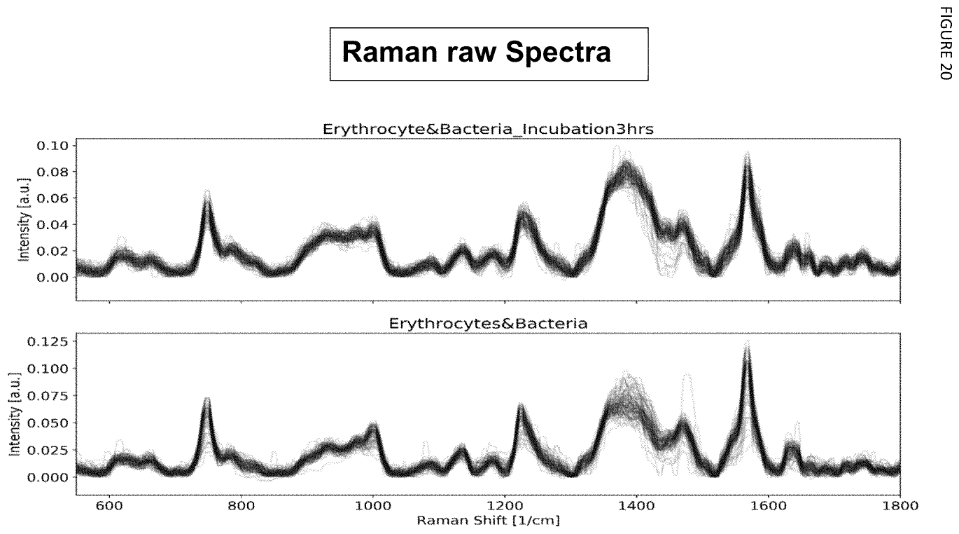

For the fast identification of contaminated blood products Raman data of erythrocytes spiked with bacteria (time 0; control) and erythrocytes incubated with bacteria for 3 hrs incubation were obtained (see FIG. 20).

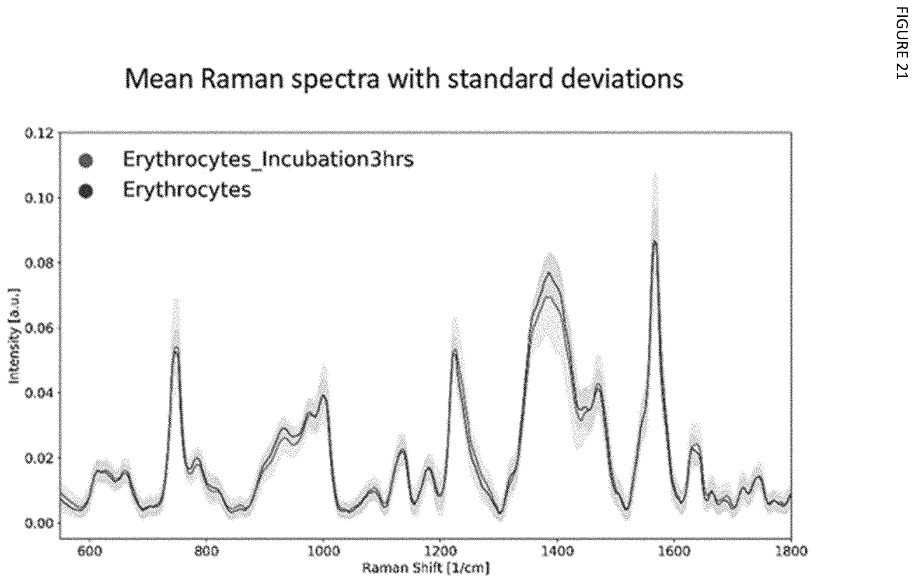

As can be derived from FIG. 21, the Raman spectra at time 0 (control) produce different mean Raman spectra as the erythrocytes incubate with bacteria for 3 hrs.

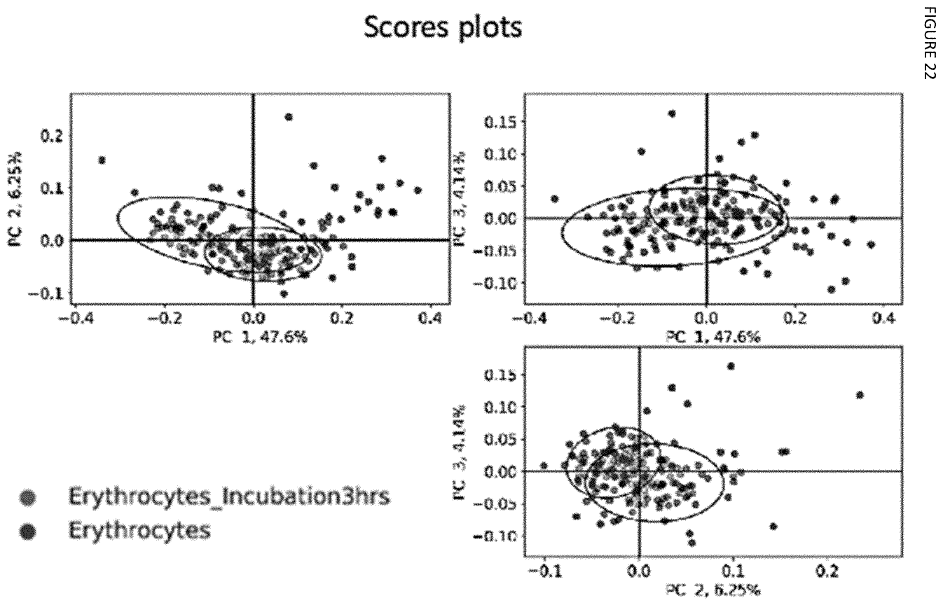

According to a principal component analysis (PCA) of the Raman data as derivable from FIG. 22 a clear difference between the control and erythrocytes after 3 hrs incubation with bacteria can be observed.

According to FIG. 23 the PC scores illustrate the difference of the Raman bands that were used for classifications.

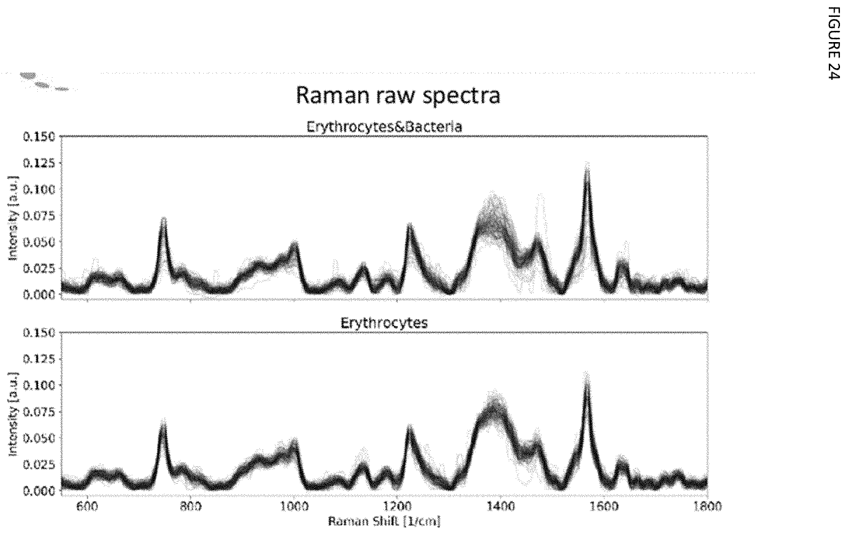

For the fast identification of contaminated blood products Raman data of erythrocytes and bacteria were compared (see FIG. 24).

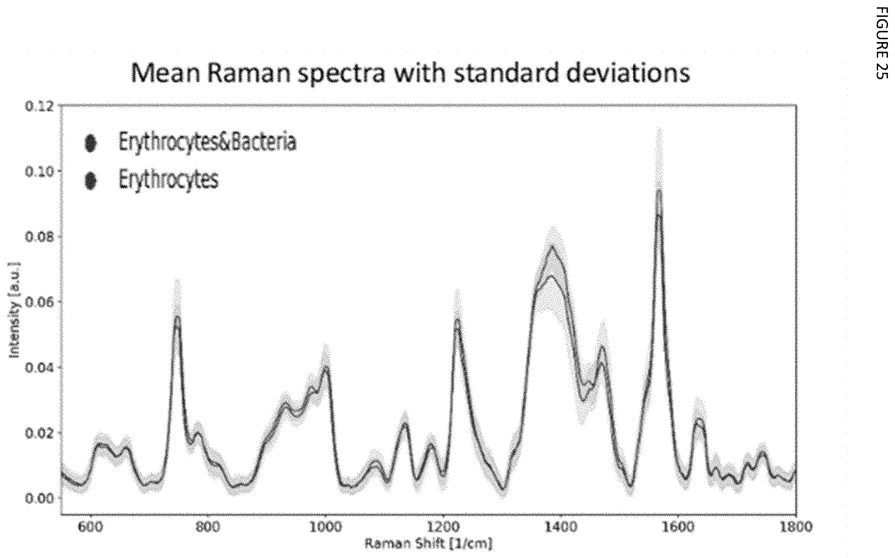

As can be derived from FIG. 25, the erythrocytes and bacteria also produce different mean Raman spectra.

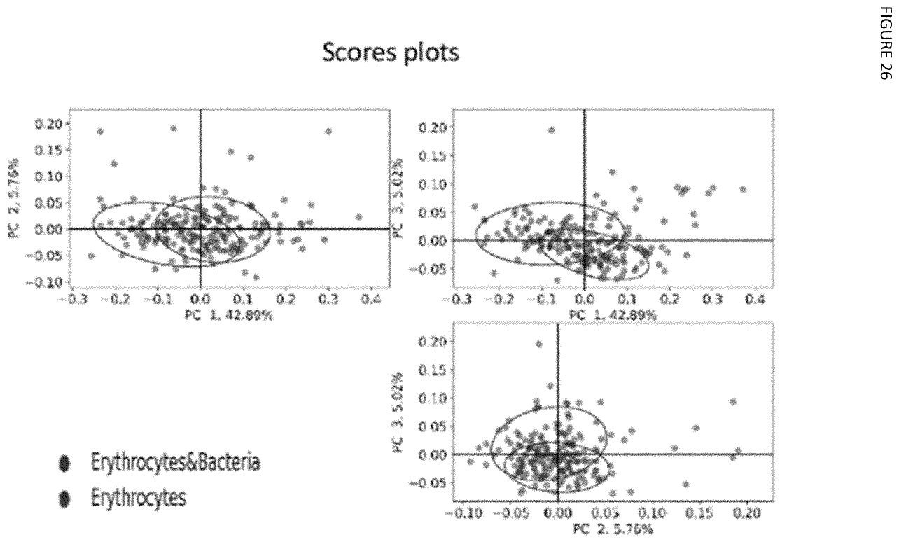

According to a principal component analysis (PCA) of the Raman data as derivable from FIG. 26 a clear difference between erythrocytes and bacteria can be observed.

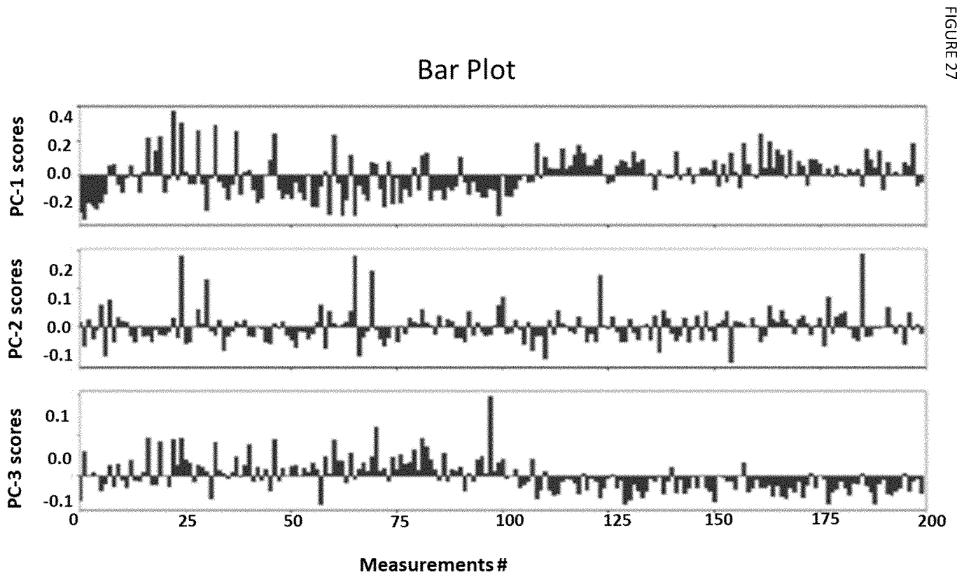

This difference is confirmed by bar plots for PC scores of FIG. 27.

### Example 5

### Detection of Airborne Microorganisms

An Agar plate was opened for 3 minutes within a room and grown for 24 hrs at room temperature.

Different colonies were scraped off and diluted in PBS buffer.

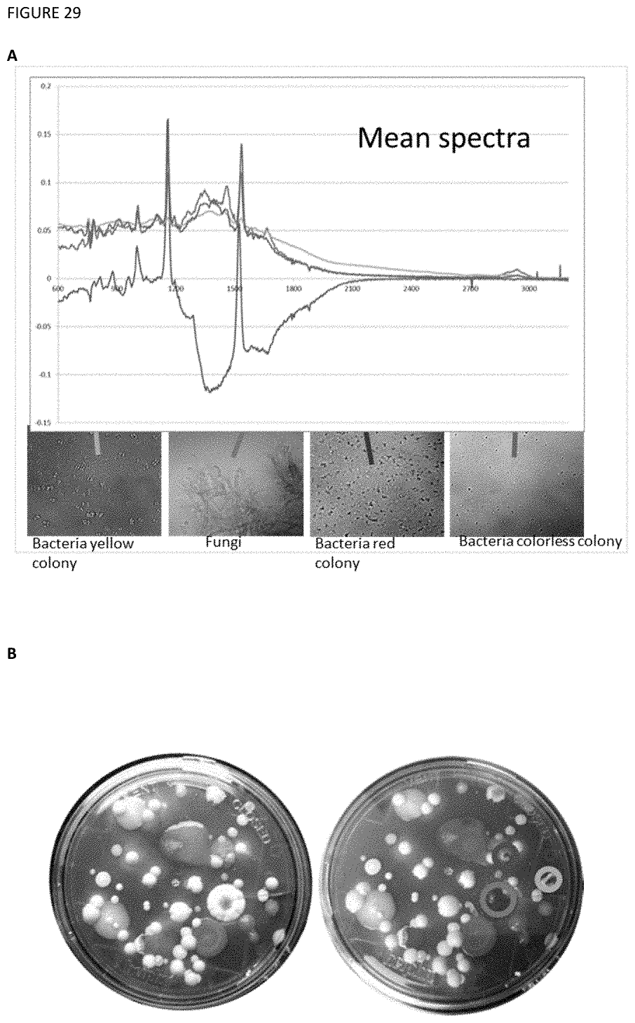

Raman mean spectra show clear differences between the different colonies (see FIG. 29).

The two dominant peaks around 1200 and 1500 wave numbers represent carotinoids (see FIG. 29A).

### Example 6

### Discrimination of Staphylococcus aureus and Staphylococcus epidermidis

Different strains Staphylococcus aureus and Staphylococcus epidermidis were collected from colonies, diluted in PBS and Raman spectra were measured.

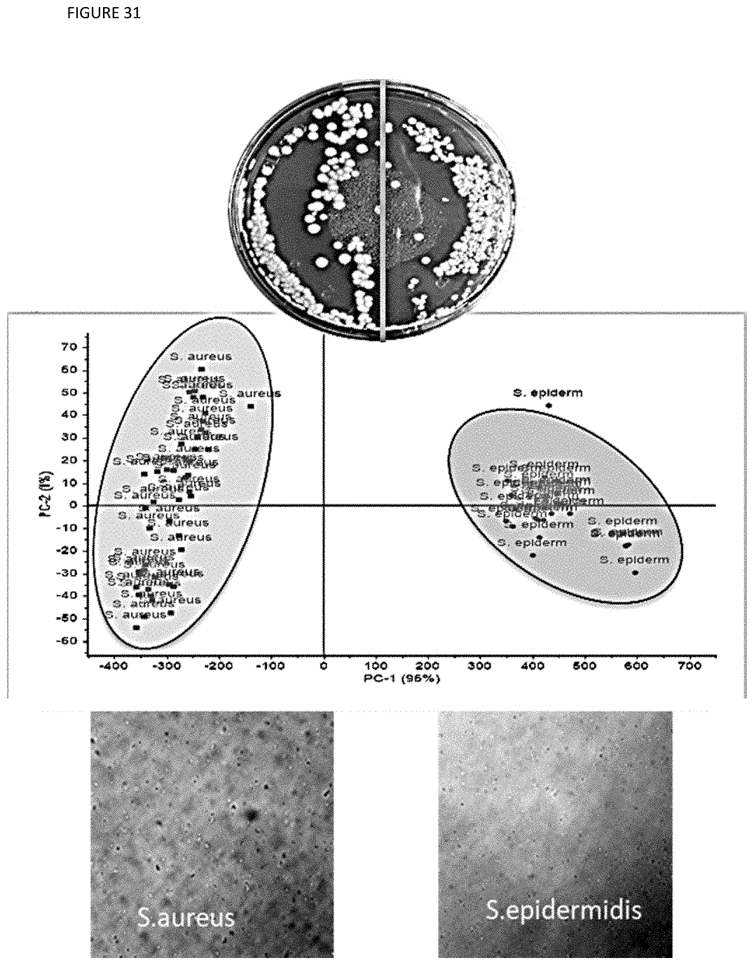

Principal component analysis of Raman spectral data was performed (see FIG. 31).

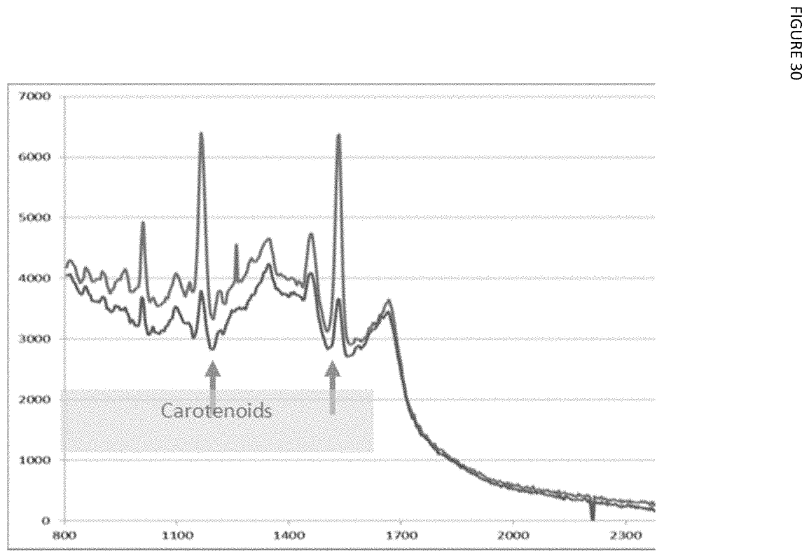

Main peaks within the mean spectra represent carotinoids (see FIG. 30)

### Example 7

### Discrimination of Pseudomonas and Staphylococcus Strains

Raman spectra of different strains of Pseudomonas (P-50BA, P-52BA, P-80BA) and Staphylococcus (S-404 and S-407) were measured and compared.

Principal component analyses of Raman spectral data were performed.

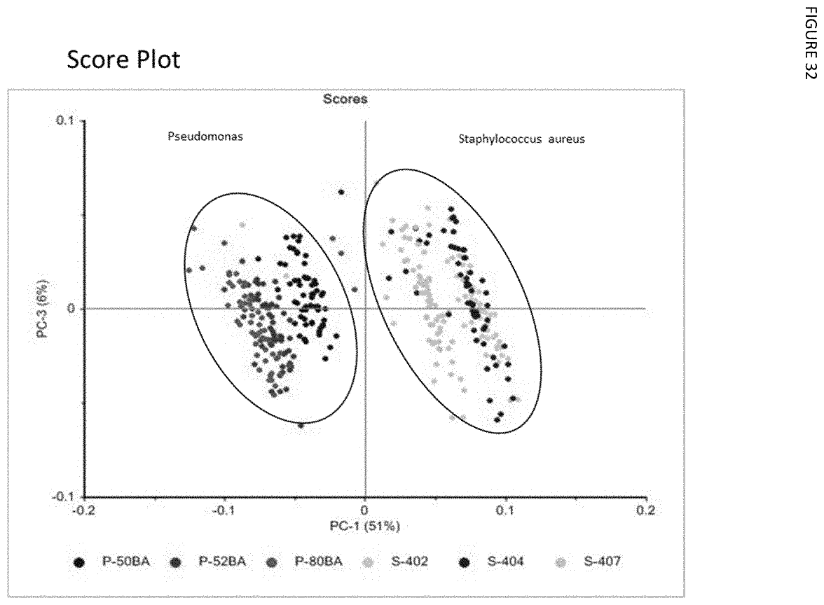

Score plots demonstrate that the different bacteria species are assembling in clearly distinct clusters (see FIG. 32).

In addition within the two different clusters the subclasses (bacterial strains) also differ clearly (see FIG. 32).

### Example 7

### Raman Spectra of S. aureus and EHEC Bacteria

EHEC bacteria as well as Staphylococcus aureus bacteria were collected from colonies, diluted in PBS and Raman spectra were measured.

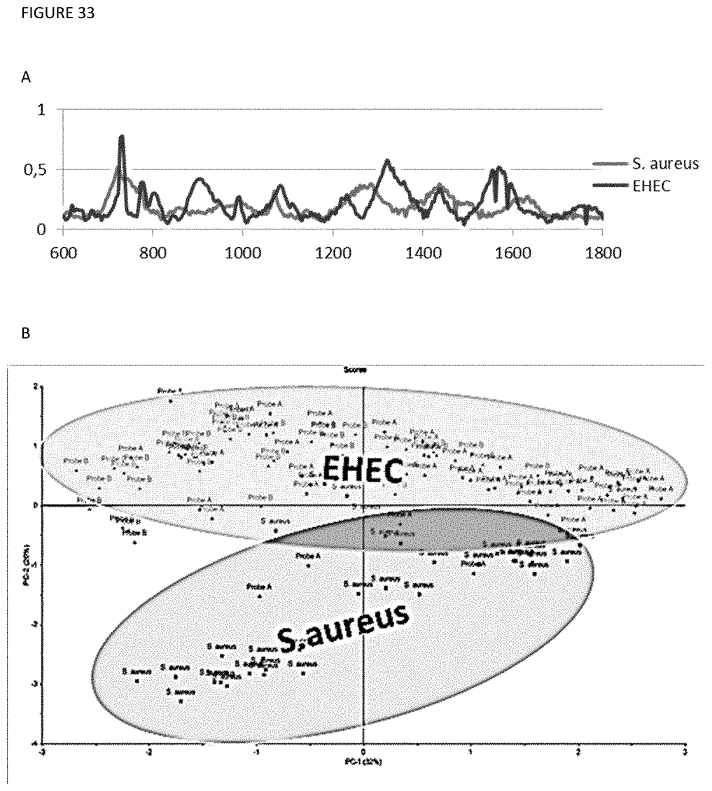

Mean spectra of S. aureus and EHEC were obtained and principal component analyses were performed. Differences are clearly visible in FIG. 33.

### Example 8

### Discrimination of EHEC Strains

Different strains of EHEC Bacteria—EHEC 52371 and EHEC 5756—were collected from colonies, diluted in PBS and Raman spectra were measured.

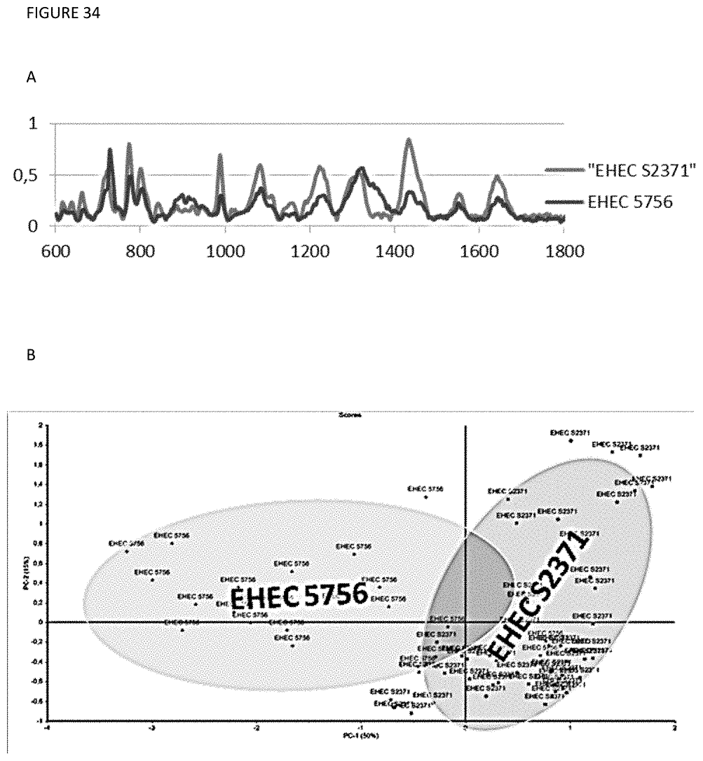

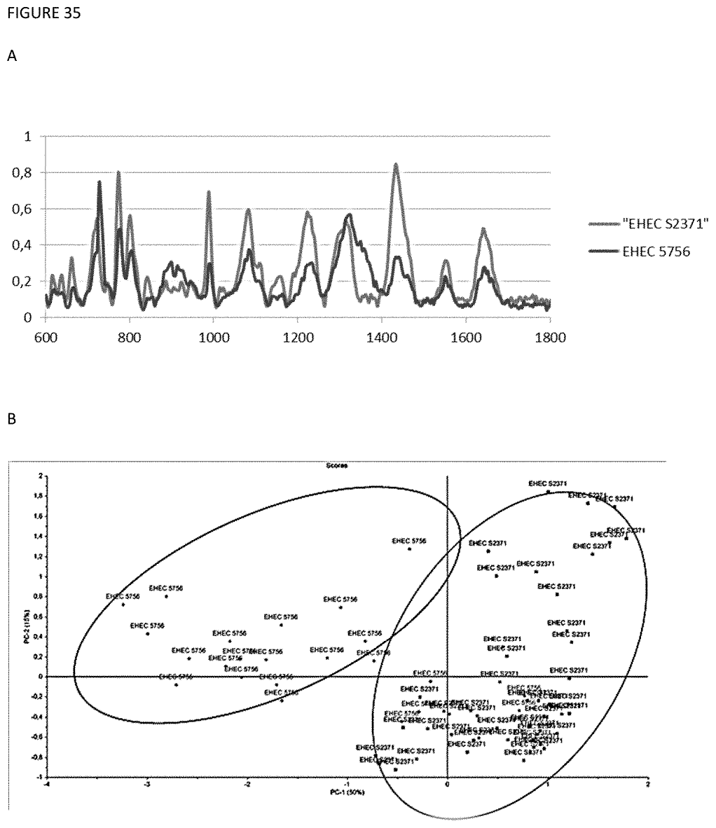

Mean Spectra of EHEC 52371 and EHEC 5756 are shown in FIG. 34A and FIG. 35A.

Principal component analysis of Raman spectral data was performed. The results are depicted in FIG. 34B and FIG. 35B.

As can be derived from FIGS. 34 and 35 a clear discrimination between EHEC strains could be demonstrated.

### Example 9

### Discrimination of Three Different E. coli Strains

Different Escherichia coli strains were grown in full medium for 16 hrs at 37° C.

Bacteria samples were pipetted into the channels of a Channel Slide and Raman spectra of single bacteria were measured using simultaneous trapping forces.

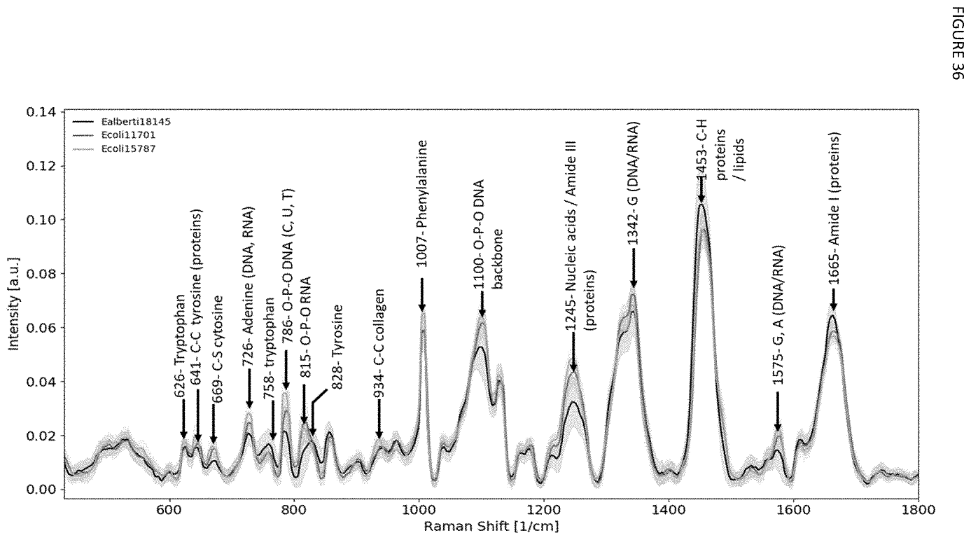

Mean spectra of Escherichia coli 11701 and Escherichia coli 15787 and Escherichia alberti 18145 differ in several peaks as can be derived from FIG. 36.

Discriminating peaks are marked with an arrow and described including wavenumber and corresponding biomolecules such as tryptophane, tyrosins, cytosins.

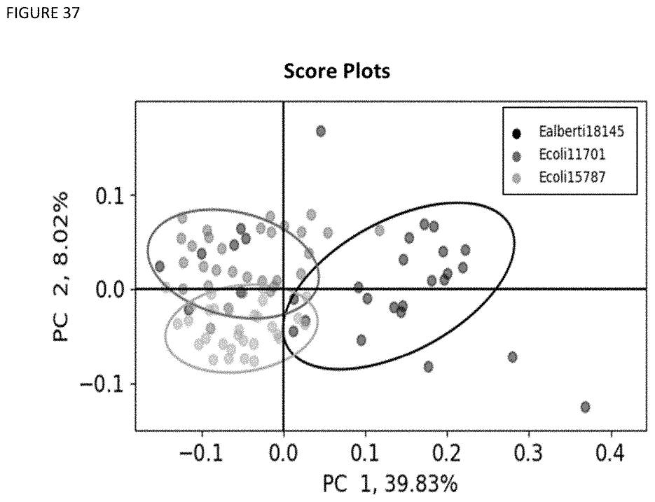

Score plots of the different bacterial strains (E. coli 11701 and E. coli 15787 and E. alberti 18145) show three clearly distinct clusters with only minimal overlap (see FIG. 37).

### Example 10

### Effect of Different Medium Conditions on E. coli 15787

Escherichia coli strain 15787 was grown under hunger stress and compared to the same strain grown on full-agar.

Both samples were grown for 16 hrs at 37° C. and washed in PBS prior to fixation in PFA.

Bacteria samples were pipetted into the channels of a Channel Slide and Raman spectra of single bacteria were measured using simultaneous trapping forces. Laser trapping arrested the bacteria within the Raman-Laser Focus and allowed to provide reliable Raman spectral results.

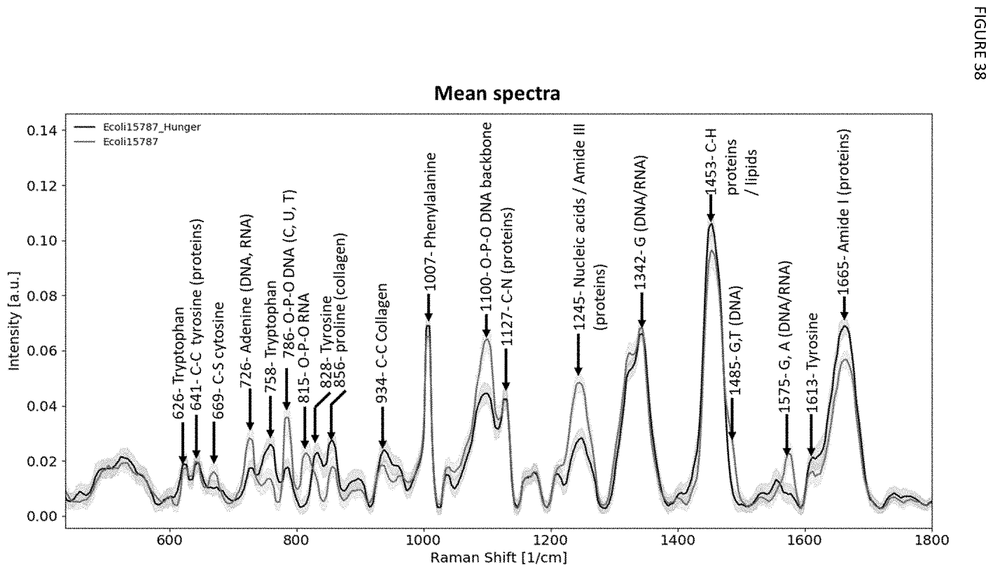

Mean spectra of Escherichia coli (E. coli 15787) in normal vs hunger medium clearly differ in several peaks. Discriminating peaks are marked with an arrow and described including wavenumber and corresponding biomolecules such as tryptophane, tyrosins and cytosins (see FIG. 38).

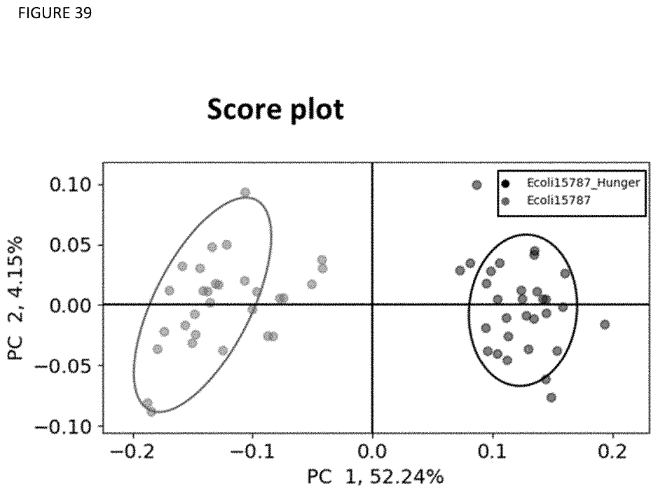

Score Plot of Escherichia coli (E coli 15787) grown in normal vs E coli 15787 grown in hunger medium clearly depict two distinct clusters (see FIG. 39).

### Example 11

### Experiments with Listeria Strains

Listeria were cultivated in normal medium and under routine conditions. One group was killed by heating (cooking) them at 90° C. Bacteria samples were pipetted into the channels of a Channel Slide and Raman spectra of single bacteria were measured using simultaneous trapping forces.

Laser trapping arrested the bacteria within the Raman-Laser focus and allowed to provide reliable Raman spectral results.

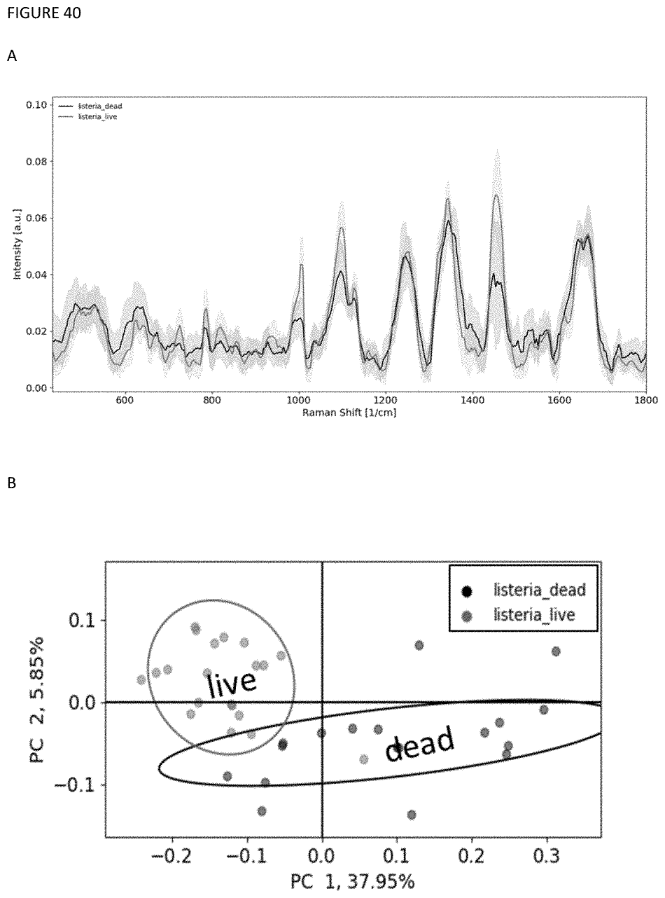

Mean spectra of the dead/live bacteria samples have peaks that clearly differ from each other and score Plot of the two samples depict clearly distinct clusters (see FIG. 40).

The possibility to discriminate dead from live bacteria is a clear advantage of Raman spectroscopy compared to PCR analysis.

1. An in vitro method for analyzing a liquid sample as to the presence, identity and properties of floating microbes comprising:
a) isolating microbes from the liquid sample by centrifugation or filtration of the liquid sample;
b) analyzing said floating microbes spectroscopically by means of spontaneous Raman spectroscopy; and
c) determining antibiotic susceptibility of said floating microbes spectroscopically by means of spontaneous Raman spectroscopy.

2. (canceled)

3. The method of claim 1, wherein said filtration is performed in a chip designed to size-exclude components within the liquid sample which are larger than microbes.

4. The method of claim 3, wherein the microbes are enriched in a micro-chamber of the chip.

5. The method of claim 3, wherein said chip is a part of a microfluidic system.

6. The method of claim 1, additionally comprising as step a-(i) a quantification of the isolated microbes, wherein the quantification is performed by means of image analysis of isolated microbes within a channel of the chip.

7. (canceled)

8. The method of claim 1, wherein steps b) and c) comprise recording at least one Raman spectrum by means of Raman spectroscopy of an isolated microbe.

9. The method of claim 8, wherein the analysis of step b) and the determination of step c) comprises collecting and arresting a microbe in an optical trap in order to record a Raman spectrum,
wherein said optical trapping forces are produced simultaneously by means of an excitation beam of a Raman spectroscopy system; or
wherein the analysis of step b) and the determination of step c) comprises collecting, slowing down movement and arresting a microbe in a decelerating material such as a fibrous gel, a hydrogel or collagen gel in order to record a Raman spectrum.

10. (canceled)

11. (canceled)

12. The method of claim 8, wherein step b) comprises a comparison of the Raman spectrum obtained from the microbe isolated in step a) with a reference spectrum, preferably derived from a database, thereby determining the identity of said microbe.

13. The method of claim 1, wherein said determination of antibiotic susceptibility of the microbes in step c) comprises obtaining a Raman spectrum for a microbe prior and subsequent to the exposure of the microbe to a single antibiotic or to a combination of at least two different antibiotics simultaneously or sequentially, preferably in the form of a gradient of said antibiotic or said combination of antibiotics,
preferably, wherein said microbe is exposed to the antibiotic for about 0.5 to 30 minutes.

14. (canceled)

15. (canceled)

16. (canceled)

17. The method of claim 8, wherein the method comprises conducting a statistical evaluation of the at least one Raman spectrum,
preferably a principal component analysis and/or cluster analysis and/or a linear discriminant analysis (LDA) of the at least one Raman spectrum.

18. (canceled)

19. (canceled)

20. The method of claim 8, wherein the method comprises statistical evaluation and judgment on the basis of artificial intelligence and/or machine learning algorithms for complex matrix data evaluation.

21. (canceled)

22. (canceled)

23. The method of claim 1, wherein the method is for the detection of sepsis in a subject, preferably for the detection of sepsis and antibiotic susceptibility of the sepsis' causative agents.

24. A device for analyzing a liquid sample as to the presence, identity and properties of microbes, wherein the device comprises as a first unit (i) a chip comprising a filtering unit and an antibiotics exposure unit capable of determining the susceptibility of microbes to an antibiotic; as a second unit (ii) a Raman spectroscopy system; and as a third unit (iii) an evaluation module which is coupled to the Raman spectroscopy system, wherein said evaluation module is preferably designed to perform principal component analysis and/or a normalization on specific band and/or cluster analysis and/or LDA analysis, more preferably wherein said evaluation module is configured to analyse isolated microbes by comparing the Raman spectrum obtained from an isolated microbe with a reference spectrum such as a reference spectrum derived from a database.

25. The device of claim 24, wherein said device comprises as a forth unit (iv) a microfluidic component for semi-automated measurement of microbes and/or for transporting microbes and/or for separating said liquid sample components or microbes, which is coupled to the Raman spectroscopy system.

26. The device of claim 24, wherein said device further comprises an integrated optical trapping module.

27. The device of claim 24, wherein said filtering unit of the chip is designed to size-exclude components within the liquid sample which are larger than microbes, thereby isolating said microbes.

28. The device of claim 24, wherein said antibiotics exposure unit of the chip comprises one or more micro-chambers comprising an antibiotic or a combination of antibiotics, wherein said antibiotic or said combination of antibiotics is preferably lyophilized.

29. (canceled)

30. (canceled)

31. (canceled)

32. The device of claim 24, which is configured to perform the method of claim 1.

33. A system comprising the device of claim 24 and a module comprising a database comprising reference values of Raman spectra obtained from microbes.

34. Use of the method of claim 1, of the device of claim 24 or of the system of claim 33 for the detection of sepsis in a subject, preferably for the detection of sepsis and antibiotic susceptibility of the sepsis' causative agents.

35. (canceled)

36. (canceled)
